(19) United States
(12) Patent Application Publication
(10) Pub. No.: US 2019/0361034 A1
(43) Pub. Date: Nov. 28, 2019

(54) BINDING ASSAY

(71) Applicant: Immutep S.A.S., Orsay (FR)

(72) Inventors: Min CHEN, Shanghai (CN); Justin Xiaoqing JIA, Shanghai (CN)

(21) Appl. No.: 16/471,105

(22) PCT Filed: Dec. 18, 2017
(86) PCT No.: PCT/CN2017/116889

§ 371 Date: Jun. 19, 2019

(30) Foreign Application Priority Data
Dec. 19, 2016 (CN) 201611180971.4

(51) Int. Cl. G01N 33/68; G01N 21/45

(52) U.S. Cl. CPC G01N 33/6857; G01N 2021/772; G01N 2021/458; G01N 21/45

(57) ABSTRACT

Methods for determining MHC class II binding activity of a preparation comprising lymphocyte activation gene-3 (LAG-3) protein, or a fragment, derivative, or analogue thereof, is described. The methods comprise determining binding of the LAG-3 protein, fragment, derivative, or analogue to MHC class II molecules using bio-layer interferometry (BLI). Such methods can be used as a quality control assay in good manufacturing practice (GMP) grade production of such compounds. Probes and kits for carrying out the methods are also described.

This invention relates to methods for determining MHC class II binding activity of preparations of lymphocyte activation gene-3 (LAG-3) protein, or fragments, derivatives, or analogues thereof, and to probes and kits for use in the methods.

LAG-3 protein is a CD4 homolog type I membrane protein with four extracellular immunoglobulin superfamily domains. Similar to CD4, LAG-3 oligomerizes at the surfaces of T cells and binds to MHC class II molecules on antigen-presenting cells (APCs) but with significantly higher affinity than CD4. LAG-3 is expressed on activated CD4⁺ and CD8⁺ T lymphocytes where it associates with the CD3/T cell receptor complex at the cell surface and negatively regulates signal transduction. As a consequence, it negatively regulates T cell proliferation, function, and homeostasis. LAG-3 is upregulated on exhausted T cells compared with effector or memory T cells. LAG-3 is also upregulated on tumor infiltrating lymphocytes (TILs), and blockade of LAG-3 using anti-LAG-3 antibody can enhance anti-tumour T cell responses.

IMP321 is a recombinant soluble LAG-3Ig fusion protein that binds to MHC class II with high avidity. It is a first-in-class immunopotentiator targeting MHC class II-positive antigen-presenting cells (APCs) (Fougeray et al.: A soluble LAG-3 protein as an immunopotentiator for therapeutic vaccines: Preclinical evaluation of IMP321. Vaccine 2006, 24:5426-5433; Brignone et al.: IMP321 (sLAG-3) safety and T cell response potentiation using an influenza vaccine as a model antigen: A single-blind phase I study. Vaccine 2007, 25:4641-4650; Brignone et al.: IMP321 (sLAG-3), an immunopotentiator for T cell responses against a HBsAg antigen in healthy adults: a single blind randomised controlled phase I study. J Immune Based Ther Vaccines 2007, 5:5; Brignone et al.: A soluble form of lymphocyte activation gene-3 (IMP321) induces activation of a large range of human effector cytotoxic cells. J Immunol 2007, 179:4202-4211). IMP321 has been tested in previously-treated advanced renal cell carcinoma patients known to be immunosuppressed and shown to induce an increase in the percentage of circulating activated CD8 T cells and of long-lived effector-memory CD8 T cells in all patients treated by repeated injections over 3 months, without any detectable toxicity (Brignone et al.: A phase I pharmacokinetic and biological correlative study of IMP321, a novel MHC class II agonist in patients with advanced renal cell carcinoma. Clin Cancer Res 2009, 15:6225-6231). A concentration of only a few ng/mL IMP321 has been shown to be active in vitro on APC, showing the great potency of IMP321 as an agonist of the immune system (Brignone, et al., 2009, supra).

In a study in metastatic breast carcinoma (MBC) patients, Brignone et al. (First-line chemoimmunotherapy in metastatic breast carcinoma: combination of paclitaxel and IMP321 (LAG-3Ig) enhances immune responses and antitumor activity. Journal of Translational Medicine 2010, 8:71) demonstrated that IMP321 expanded and activated for several months both the primary target cells (MHC class II-positive monocytes/dendritic cells) to which IMP321 binds, and the secondary target cells (NK/CD8+ effector memory T cells) which are activated subsequently. By pooling results from all 30 patients and comparing tumor regression with an appropriate historical control group, they saw a doubling of the objective response rate which suggests that IMP321 is a potent agonist of effective anti-cancer cellular immune responses in this clinical setting.

WO 99/04810 describes use of LAG-3 protein, or fragments or derivatives thereof, as an adjuvant for vaccination, and in cancer treatment. Use of LAG-3 protein, or fragments or derivatives thereof, for the treatment of cancer and infectious disease is described in WO 2009/044273.

In view of the medical uses of LAG-3, and fragments or derivatives thereof, there is a need to provide preparations of such compounds that comply with good manufacturing practices (GMP). Such practices are required in order to conform to the guidelines recommended by agencies that control authorization and licensing for manufacture and sale of active pharmaceutical products. These guidelines provide minimum requirements that a pharmaceutical manufacturer must meet to assure that the products are of high quality and do not pose any risk to the consumer or public. As part of the quality control procedure in GMP-grade manufacture of proteins, it is necessary to determine whether preparations of such compounds retain a high level of bioactivity.

We have found, however, that several conventional methods for determining protein-protein interactions are not suitable for determining specific binding of the LAG-3 derivative IMP321 to MHC class II molecules expressed on the surface of immune cells. In particular, fluorescence-activated cell sorting (FACS) was not suitable for distinguishing IMP321 preparations with differing abilities to bind to MHC class II-expressing cells. No upper plateaus were observed at increasing concentrations of IMP321 for the binding curves obtained using FACS. This prevents calculation of the relative potencies of different preparations, which requires converged plateaus (parallelism).

We have also found that IMP321 binds non-specifically to plates used for MesoScale Discovery (MSD) electrochemiluminescent (ECL) assays, and Enzyme-Linked Immunosorbent Assays (ELISAs). Whilst non-specific binding of IMP321 to plates used for ELISA and MSD assays was dramatically reduced by use of casein as a blocking reagent, this lowered the absolute signal in the MSD assay. No upper plateaus were observed for binding curves obtained using assays in which cells expressing MHC class II molecules were immobilised to the MSD plates. A different ELISA technique was also tested, in which cells expressing MHC class II molecules were transferred to another plate after binding of IMP321, in order to minimise the effect of non-specific binding of IMP321 to the plates. However, the well-to-well signal variation was found to be unacceptable. In view of this, it was concluded that neither MSD ECL assays nor ELISA assays could be used to determine specific binding of IMP321 to the immobilised cells in a quality control assay to test GMP-grade product.

There is a need, therefore, to provide a method for determining MHC class II binding activity of preparations of LAG-3 protein, or fragments, derivatives, or analogues thereof, which is suitable for use as a quality control assay in GMP-grade production of such compounds.

According to the invention, there is provided a method for determining MHC class II binding activity of a preparation comprising lymphocyte activation gene-3 (LAG-3) protein, or a fragment, derivative, or analogue thereof, wherein the method comprises determining binding of the LAG-3 protein, fragment, derivative, or analogue to MHC class II molecules using bio-layer interferometry (BLI).

The term “bio-layer interferometry (BLI)” is used herein to refer to a fibre-optic assay based on phase-shift interferometry, for example as described in U.S. Pat. No. 5,804,453 (Chen). Developments to the BLI technique, including developments aimed at enhancing the sensitivity and accuracy of analyte detection, are described in WO 2005/047854 and WO 2006/138294 of ForteBio, Inc.

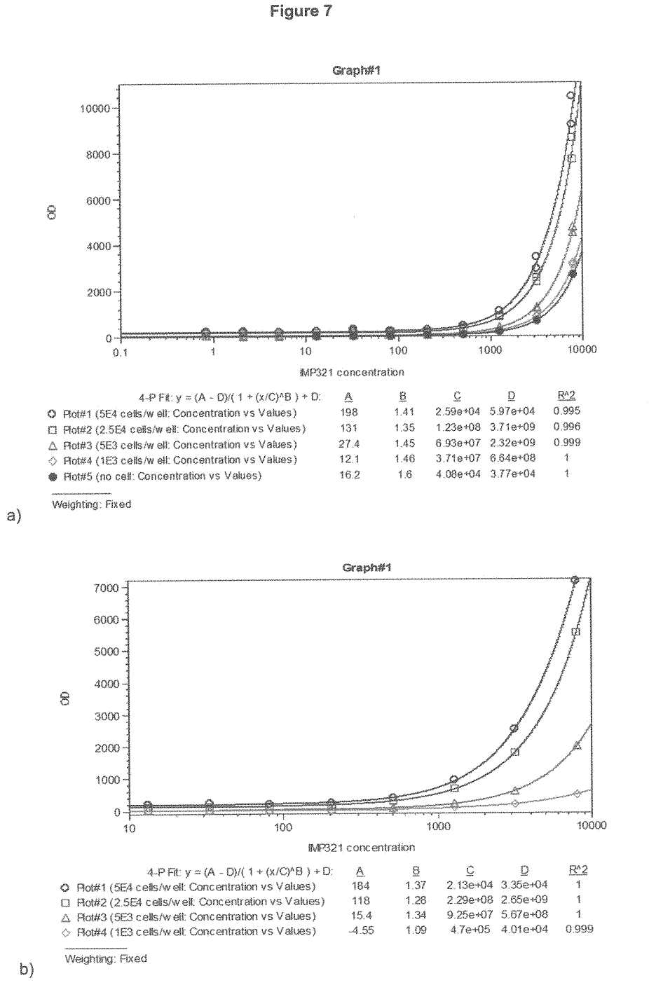

U.S. Pat. No. 5,804,453 describes a probe, method, and system for detecting analyte binding to a fibre-optic end surface. Analyte detection is based on a change in the thickness at the end surface of the optical fibre resulting from the binding of analyte molecules to the surface, with greater amount of analyte producing a greater thickness-related change in the interference signal. The change in interference signal is due to a phase shift between light reflected from the end of the fibre and from the binding layer carried on the fibre end, as illustrated particularly in FIGS. 7a and 7b of U.S. Pat. No. 5,804,453.

The probe described in U.S. Pat. No. 5,804,453 includes a fibre optic section having a proximal end tip and a distal end tip and a reagent layer disposed on the distal end tip. The reagent layer reacts (or bonds) with the substance (analyte) being detected. The fibre optic section has a first index of refraction and the reagent layer has a second index of refraction. When any of the substance bonds to the reagent layer, a resulting layer including the reagent layer and the substance is formed. The resulting layer can be treated as having a homogeneous index of refraction.

The method permits the concentration of a substance in a sample solution to be determined using the fibre optic probe. The method includes steps of (i) immersing the distal end of the fibre optic probe into the sample solution, (ii) optically coupling a light source with the proximal end of the fibre optic probe, (iii) detecting at least a first light beam reflected from an interface between the distal end surface of the fibre optic section and the reagent layer, and a second light beam reflected from an interface between the reagent layer and the sample solution, reflected from the distal end of the fibre optic probe, (iv) detecting an interference pattern formed by the first and second light beams at a first time, (v) detecting an interference pattern formed by the first and second light beams at a second time, and (vi) determining whether the substance is present in the sample solution based on whether a shift occurs in the interference patterns. The concentration of the substance may be determined based on a shift in the interference patterns and based on a differential between the first and second times.

The system for detecting the concentration of a substance in a sample solution has a light source for providing a light beam, a fibre optic probe, a detector, a fibre optic coupler, a fibre optic connector, and a processor. The fibre optic coupler includes a first fibre optic section having a proximal end for receiving an incident light beam, a second fibre optic section having a proximal end for delivering the reflected interference light beam to the detector, and a third fibre optic section having a distal end for connecting to the fibre optic probe. The fibre optic probe includes a proximal end for connecting to the fibre optic coupler, and a distal end tip with a reagent layer disposed thereon. The fibre optic probe produces at least a first reflected beam and a second reflected beam from the incident light beam. The detector detects an interference pattern formed by the first and second reflected beams. The coupler optically couples the light source with the fibre optic probe and optically couples the fibre optic probe with the detector. The processor determines a phase associated with an interference pattern detected by the detector at a first time, determines a phase associated with an interference pattern detected by the detector at a second time, and determines the concentration of the substance based on a shift in the phases associated with the interference patterns detected by the detector at the first and second times.

We have appreciated that the BLI technique can be used to determine the MHC class II binding activity of preparations of LAG-3 protein, or fragments, derivatives, or analogues thereof, and that such methods are particularly useful as a quality control assay in GMP-grade production of such compounds.

In particular embodiments, methods of the invention comprise determining binding of the LAG-3 protein, fragment, derivative, or analogue, to MHC class II molecules present on MHC class II-expressing cells. In such embodiments, the LAG-3 protein, fragment, derivative, or analogue may be immobilised to a reagent layer of a BLI probe, and the MHC class II-expressing cells are in solution.

The probe, method, and system described in U.S. Pat. No. 5,804,453 may be used in accordance with the present invention for determining the MHC class II binding activity of a preparation of LAG-3 protein, or a fragment, derivative, or analogue thereof, as exemplified below by binding of the recombinant LAG-3 protein derivative IMP321 to MHC class II-expressing Raji cells.

Referring to Figure a below, a biosensor probe 100 includes an optical fibre 102, and a reagent layer 104, comprising a blocking reagent (e.g. BSA) and IMP321, at a distal tip of the optical fibre 102. The blocking reagent and IMP321 may be bound to the tip of the optical fibre 102 by soaking the tip in a solution having a predetermined concentration of IMP321, or the blocking reagent, for a predetermined period.

An incident light beam 110 is sent through the optical fibre 102 toward its distal end. At the interface 106 defined between the optical fibre 102, which has a first index of refraction, and the reagent layer 104, which has a second index of refraction, a first portion 112 of the incident light beam 110 will be reflected, while a second portion 114 of the incident light beam 110 will continue through the reagent layer 104. Typically, the blocking reagent and IMP321 will be small relative to the wavelength of the incident light beam 110, from an optical perspective, so the blocking reagent and the IMP321 can be treated as forming a single reagent layer 104. At an interface 108 defined at the exposed surface of the reagent layer 104, of the second portion 114 of the incident beam 110, a first portion 116 will be reflected, while a second portion 118 will pass into the adjacent medium. Of the first portion 116 of the second portion 114 of the incident beam 110, a first portion 160 will be transmitted back through the optical fibre 102, while a second portion (not shown) will be reflected at the interface 106 back into the reagent layer 104.

At a proximal end of the optical fibre 102, the reflected beams 112 and 160 are detected and analysed. At any given point along the optical fibre 102, including its proximal end, the reflected beams 112 and 160 will exhibit a phase difference. Based on this phase difference, the thickness S₁ of the reagent layer 104 can be determined.

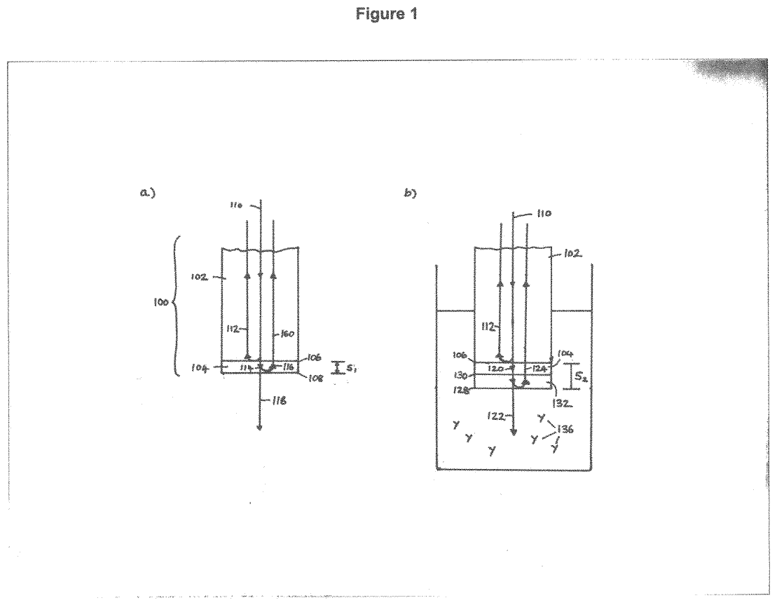

Referring to FIG. 1b below, the probe 100 is immersed in a solution 134 containing Raji cells 136 to determine binding of the cells to the immobilised IMP321. The cells 136 will bind to the immobilised IMP321 in the reagent layer 104, thereby forming a cell layer 132 over a period of time. The thickness S₂ of the layer will be a function of the time of immersion of the probe 100 in the sample fluid 134, as well as the concentration of the cells 136 in the sample fluid 134. Other molecules 138 (not shown) in the sample solution will not bind to the reagent layer 104.

The total thickness S₂ of this combined layer will be greater than the thickness S₁ of the reagent layer 104 alone. Thus, similar to the probe 100 of FIG. 1a, when an incident beam 110 is directed towards the distal tip of the optical fibre 102, at the interface 106 between the optical fibre 102 and the combined layer, a first portion 112 of the incident beam 110 is reflected, while a second portion 120 of the incident beam 110 continues through the combined layer. When the second portion 120 reaches the cells of the cell layer 132, a first portion of it (not shown) will be reflected when it meets the cellular membrane and cytoskeletal structures of the cells.

At a second interface 128 between the combined layer and the sample solution 134, a second portion 124 of the second portion 120 of the incident beam 110 is reflected, while a third portion 122 of the second portion 120 of the incident beam 110 continues through the sample solution 134. Of the second portion 124 of the second portion 120 of the incident beam 110, a first portion 126 continues back through the optical fibre 102, while a second portion (not shown) is reflected back into the combined layer at the interface 106.

At a proximal end of the optical fibre 102, the reflected beams 112 and 126 are detected and analysed. At any given point along the optical fibre 102, including its proximal end, the reflected beams 112 and 126 will exhibit a phase difference. Based on this phase difference, the thickness S₂ of the combined layer can be determined.

By determining the difference between the thickness S₂ of the combined layer and the thickness S₁ of the reagent layer 104, the thickness of the cell layer 132 can be determined. The thickness S₂ of the combined layer is determined (or “sampled”) at discrete points in time. In this way, the rate of increase of the difference between the thickness S₂ of the combined layer and the thickness S₁ of the reagent layer 104 (i.e., the rate of increase in thickness of the cell layer 132) can be determined. Based on this rate, the rate of binding of the immobilised IMP321 to MHC class II molecules on the Raji cells can be determined within a very short incubation period.

The diameter of Raji cells is approximately 5-7 μM, 1000 times the wavelength of light, so might be expected to affect the results obtained. However, the signal readout is around 1-2 nM, indicating that light is reflected near the surface of the cells. We have found that the signal change is repeatable, correlated with cell binding, and that the binding rate change is within the measurement range, so can be used to determine binding of Raji cells to IMP321 immobilised at the tip of the optical fibre.

The MHC class II binding activity of the preparation may be determined as the rate of binding of the LAG-3 protein, fragment, derivative, or analogue to the MHC class II molecules.

We have found that the binding rate obtained using the BLI assay depends on the density of MHC class II-expressing cells in the solution, whereas the binding rate is low and relatively flat when the density of non-MHC class II-expressing cells is increased. A higher rate, as well as a higher upper plateau of the binding curve, are obtained if the MHC class II-expressing cells are present at a density of at least 4E6/mL, preferably at least 6E6/mL or 8E6/mL.

We have found that the specificity of the BLI assay is improved when the reagent layer of the BLI probe has been pre-treated with a blocking reagent to minimise non-specific binding of the MHC class II-expressing cells to the reagent layer. Any suitable blocking reagent can be used, for example blocking reagents comprising inert protein such as albumin, for example bovine serum albumin (BSA).

The MHC class II-expressing cells may be immune cells expressing MHC class II molecules. Suitable examples include antigen-presenting cells, or cells of cell lines derived from immune cells. In particular embodiments, the MHC class II-expressing cells are B cells or cells of a B cell line, for example Raji cells.

We have found that MHC class II-expressing cells used for methods of the invention may be thawed, ready-to-use cells obtained from a frozen stock solution. Use of such cells eliminates the requirement to culture cells immediately before a method of the invention is carried out, can help to ensure reliability and reproducibility of results obtained by methods of the invention, and can also allow results obtained at different times to be compared.

Methods of the invention may comprise determining a rate of binding of the LAG-3 protein, fragment, derivative, or analogue, to the MHC class II molecules for a plurality of different concentrations of the LAG-3 protein, fragment, derivative, or analogue, and generating a dose-response curve for the rates of binding, for example as described in Example 6 below.

Methods of the invention may further comprise determining MHC class II binding activity of a reference sample of LAG-3 protein, or a fragment, derivative, or analogue thereof, by determining binding of the LAG-3 protein, fragment, derivative, or analogue of the reference sample to MHC class II molecules using BLI, under the same conditions used for determining binding of the LAG-3 protein, fragment, derivative, or analogue of the preparation, and comparing the MHC class II binding activity determined for the reference sample with the MHC class II binding activity determined for the preparation.

The MHC class II binding activity of the reference sample, at a predetermined concentration, may be set as 100% and diluted to various desired concentrations, for example to allow qualification or validation of measurements of MHC class II binding activity of a preparation comprising LAG-3 protein, or a fragment, derivative or analogue thereof, made using a method of the invention.

In some embodiments, the reference sample comprises a LAG-3 protein, or a fragment, derivative, or analogue thereof, that has been treated to reduce its MHC class II binding activity. Suitable treatments include, for example, deglycosylation (for example by treatment with a PNGase), storage at 37° C. for at least 12 days, oxidation (for example by treatment with 1% or 0.1% hydrogen peroxide), treatment with acid or alkali, or exposure to light for at least 5 days.

Example 6 below describes in detail a BLI assay for determining the MHC class II binding activity of immobilised IMP321 to Raji cells in solution.

There is also provided according to the invention a BLI probe for determining MHC class II binding activity of LAG-3 protein, or a fragment, derivative, or analogue thereof, which comprises a reagent layer to which the LAG-3 protein, or fragment, derivative, or analogue thereof, is immobilised.

There is further provided a kit for determining MHC class II binding activity of LAG-3 protein, or a fragment, derivative, or analogue thereof, which comprises a BLI probe having a reagent layer to which the LAG-3 protein, or fragment, derivative, or analogue thereof, is immobilised, and MHC class II-expressing cells.

In some embodiments, the reagent layer of the BLI probe has been pre-treated with a blocking reagent to minimise non-specific binding of the MHC class II-expressing cells to the reagent layer. Any suitable blocking reagent may be used, for example a blocking reagent comprising inert protein such as albumin, for example bovine serum albumin (BSA).

In some embodiments the MHC class II-expressing cells are frozen cells.

In some embodiments the MHC class II-expressing cells are Raji cells.

The MHC class II-expressing cells may be present at a density of at least 1E6/mL, preferably at least 4E6/mL, or 8E6/mL.

A kit of the invention may further include a reference sample, for example as described above, comprising LAG-3 protein, or a fragment, derivative, or analogue thereof. Preferably the MHC class II binding activity of the reference sample is known (for example as determined by a CCL4 release assay, described below).

Probes and kits of the invention may be used in methods of the invention.

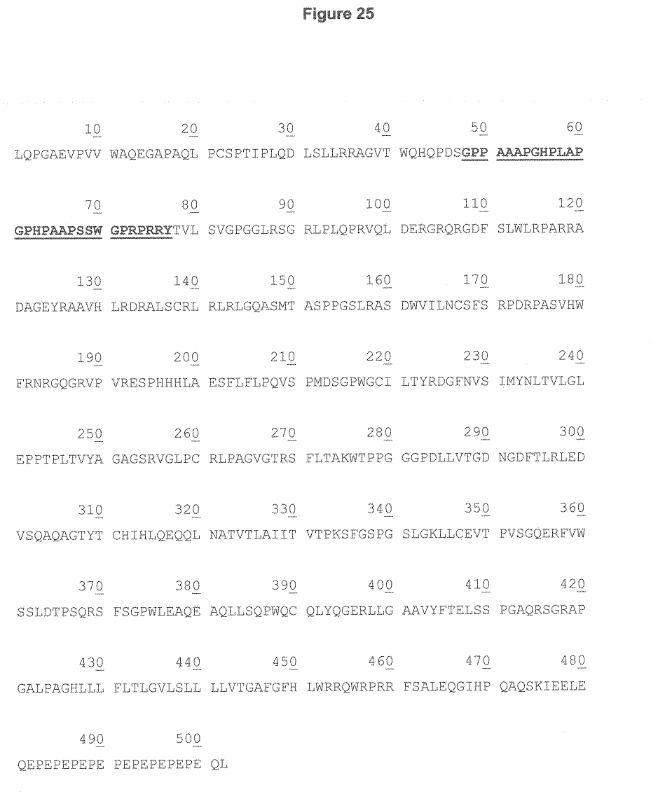

The LAG-3 protein may be an isolated natural or recombinant LAG-3 protein. The LAG-3 protein may comprise an amino sequence of LAG-3 protein from any suitable species, such as a primate or murine LAG-3 protein, but preferably a human LAG-3 protein. The amino acid sequence of human and murine LAG-3 protein is provided in FIG. 1 of Huard et al (Proc. Natl. Acad. Sci. USA, 11: 5744-5749, 1997). The sequence of human LAG-3 protein is repeated in FIG. 25 below (SEQ ID NO: 1). The amino acid sequences of the four extracellular Ig superfamily domains (D1, D2, D3, and D4) of human LAG-3 are also identified in FIG. 1 of Huard et al., at amino acid residues: 1-149 (D1); 150-239 (D2); 240-330 (D3); and 331-412 (D4).

Derivatives of LAG-3 protein include soluble fragments, variants, or mutants of LAG-3 protein that are able to bind MHC class II molecules. Several derivatives of LAG-3 protein are known that are able to bind to MHC class II molecules. Many examples of such derivatives are described in Huard et al (Proc. Natl. Acad. Sci. USA, 11: 5744-5749, 1997). This document describes characterization of the MHC class II binding site on LAG-3 protein. Methods for making mutants of LAG-3 are described, as well as a quantitative cellular adhesion assay for determining the ability of LAG-3 mutants to bind class II-positive Daudi cells. Binding of several different mutants of LAG-3 to MHC class II molecules was determined. Some mutations were able to reduce class II binding, while other mutations increased the affinity of LAG-3 for class II molecules. Many of the residues essential for binding MHC class II proteins are clustered at the base of a large 30 amino acid extra-loop structure in the LAG-3 D1 domain. The amino acid sequence of the extra-loop structure of the D1 domain of human LAG-3 protein is GPPAAAPGHPLAPGPHPAAPSSWGPRPRRY (SEQ ID NO: 2), the underlined sequence in FIG. 25.

The LAG-3 protein derivative may comprise the 30 amino acid extra-loop sequence of the human LAG-3 D1 domain, or a variant of such sequence with one or more conservative amino acid substitutions. The variant may comprise amino acid sequence that has at least 70%, 80%, 90%, or 95% amino acid identity with the 30 amino acid extra-loop sequence of the human LAG-3 D1 domain.

The derivative of LAG-3 protein may comprise an amino acid sequence of domain D1, and optionally domain D2, of LAG-3 protein, preferably human LAG-3 protein.

The derivative of LAG-3 protein may comprise an amino acid sequence that has at least 70%, 80%, 90%, or 95% amino acid identity with domain D1, or with domain D1 and D2, of LAG-3 protein, preferably human LAG-3 protein.

The derivative of LAG-3 protein may comprise an amino acid sequence of domains D1, D2, D3, and optionally D4, of LAG-3 protein, preferably human LAG-3 protein.

The derivative of LAG-3 protein may comprise an amino acid sequence that has at least 70%, 80%, 90%, or 95% amino acid identity with domain D1, D2, and D3, or with domain D1, D2, D3, and D4, of LAG-3 protein, preferably human LAG-3.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970. J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

The LAG-3 protein derivative may be fused to Immunoglobulin Fc amino acid sequence, preferably human IgG1 Fc amino acid sequence, optionally by a linker amino acid sequence.

The ability of a derivative of LAG-3 protein to bind to MHC class II molecules may be determined using a quantitative cellular adhesion assay as described in Huard et al (supra). The affinity of a derivative of LAG-3 protein for MHC class II molecules may be at least 20%, 30%, 40%, 50%, 60%, 70%, 80%, 90%, or 100% of the affinity of human LAG-3 protein for class II molecules. Preferably the affinity of a derivative of LAG-3 protein for MHC class II molecules is at least 50% of the affinity of human LAG-3 protein for class II molecules.
Examples of suitable derivatives of LAG-3 protein that are able to bind MHC class II molecules include derivatives comprising:
amino acid residues 23 to 448 of the human LAG-3 sequence;
amino acid sequence of domains D1 and D2 of LAG-3;
amino acid sequence of domains D1 and D2 of LAG-3 with an amino acid substitution at one or more of the following positions: position 73 where ARG is substituted with GLU; position 75 where ARG is substituted with ALA or GLU; position 76 where ARG is substituted with GLU; position 30 where ASP is substituted with ALA; position 56 where HIS is substituted with ALA; position 77 where TYR is substituted with PHE; position 88 where ARG is substituted with ALA; position 103 where ARG is substituted with ALA; position 109 where ASP is substituted with GLU; position 115 where ARG is substituted with ALA;
amino acid sequence of domain D1 of LAG-3 with a deletion of amino acid residues 54 to 66;
a recombinant soluble human LAG-3Ig fusion protein (IMP321)—a 200-kDa dimer produced in Chinese hamster ovary cells transfected with a plasmid encoding for the extracellular domain of hLAG-3 fused to the human IgG1 Fc. The sequence of IMP321 is given in SEQ ID NO: 17 of US 2011/0008331.

Embodiments of the invention are described below, by way of example only, with reference to the following drawings in which:

FIG. 1 shows operation of the probe used to determine MHC class II binding activity of LAG-3 protein, or fragments, derivatives, or analogues thereof, according to an embodiment of the invention (Figure taken from U.S. Pat. No. 5,804,453);

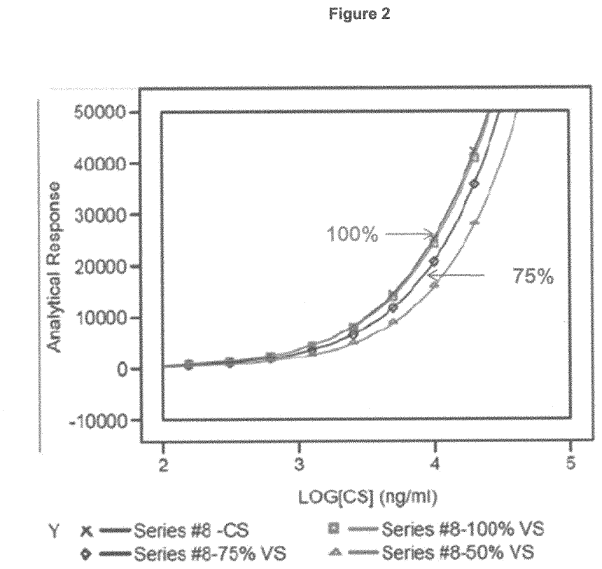

FIG. 2 shows the results of a FACS assay to determine binding of IMP321 to Raji cells;

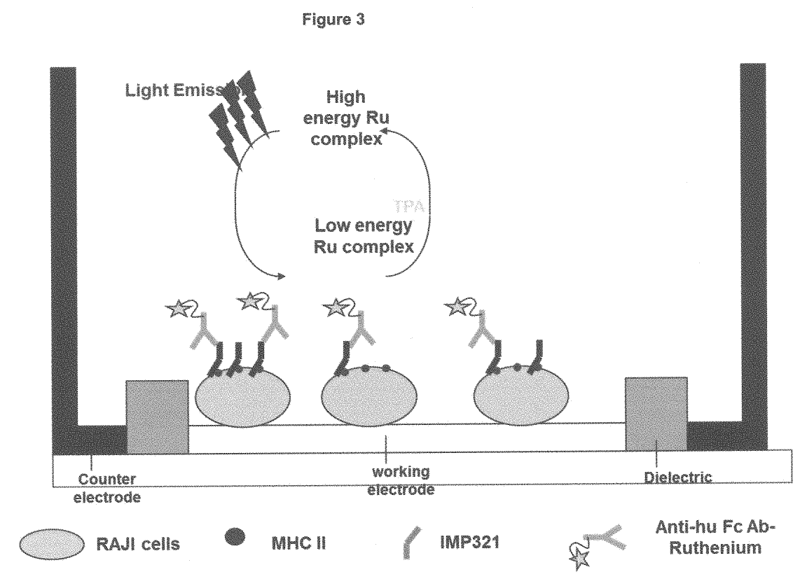

FIG. 3 shows schematically a MesoScale Discovery (MSD) electrochemiluminescent (ECL) assay to determine binding of IMP321 to Raji cells;

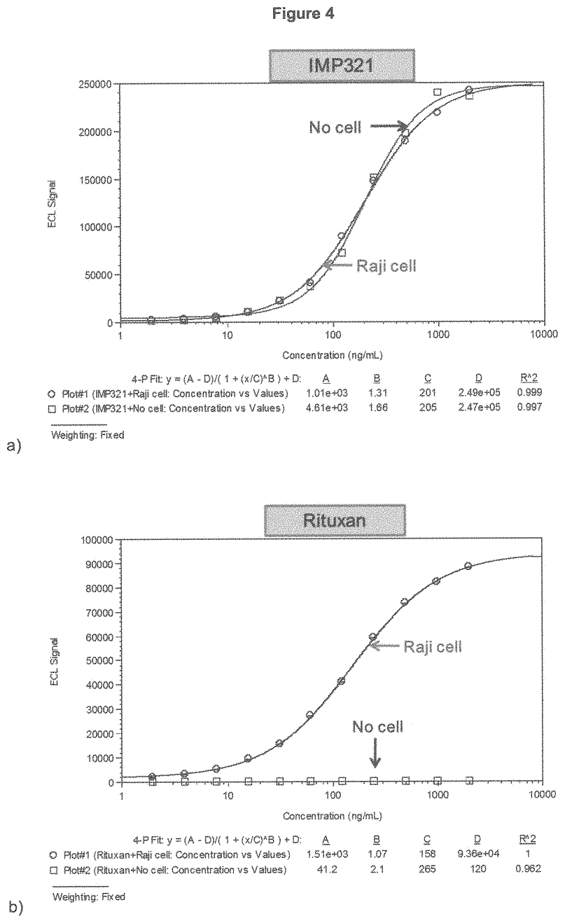

FIG. 4(a) shows a plot of the ECL signal obtained for an MSD assay at different concentrations of IMP321 in the presence and absence of Raji cells; FIG. 4(b) shows a plot of the ECL signal obtained for an MSD assay at different concentrations of Rituxan in the presence and absence of Raji cells;

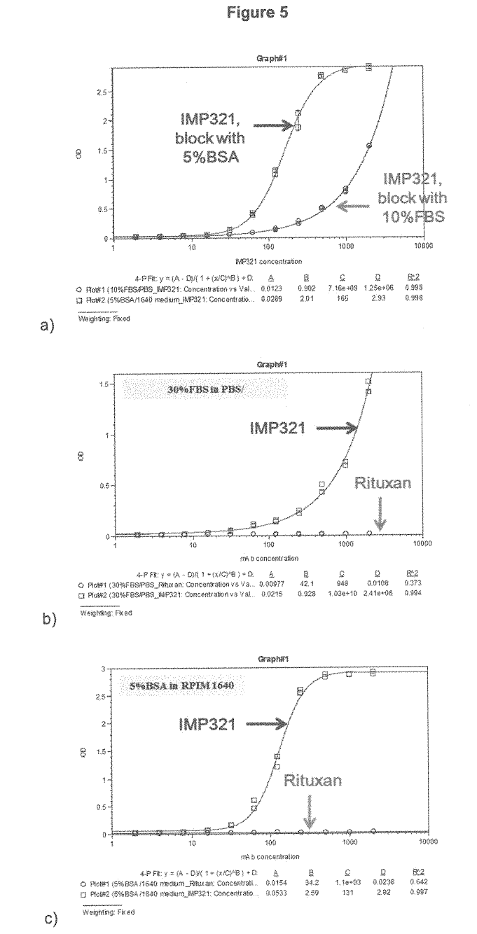

FIG. 5(a) shows a plot of the OD signal obtained for an ELISA at different concentrations of IMP321 following blocking of the ELISA plate with 5% BSA or 10% FBS; FIG. 5(b) shows a plot of the OD signal obtained for an ELISA at different concentrations of IMP321 or Rituxan following blocking of the ELISA plate with 30% FBS in PBS; FIG. 5(c) shows a plot of the OD signal obtained for an ELISA at different concentrations of IMP321 or Rituxan following blocking of the ELISA plate with 5% BSA in RPIM1640;

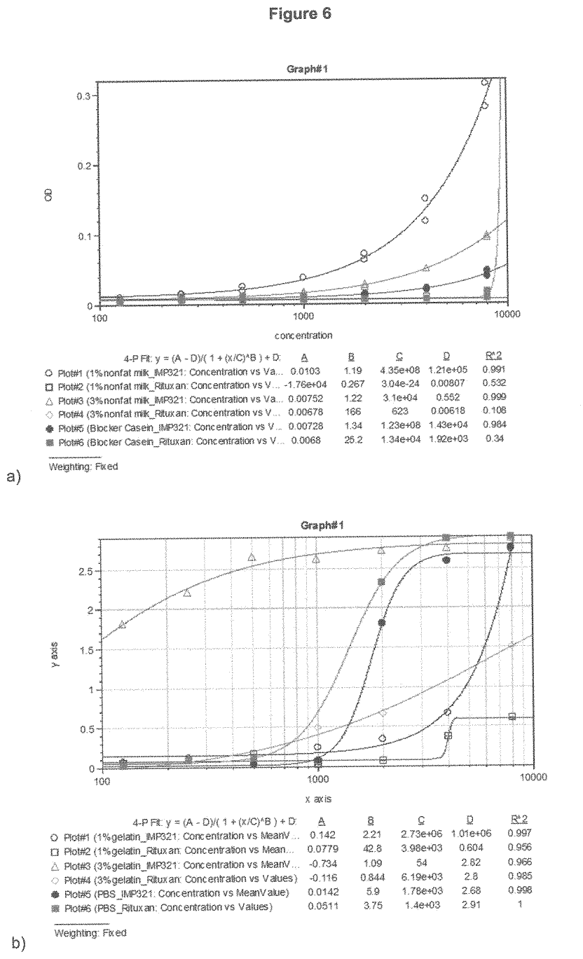

FIG. 6(a) shows a plot of the OD signal obtained for an ELISA at different concentrations of IMP321 or Rituxan following blocking of the ELISA plate with different blocking reagents (1% nonfat milk, 3% nonfat milk, Casein); FIG. 6(b) shows a plot of the OD signal obtained for an ELISA at different concentrations of IMP321 or Rituxan following blocking of the ELISA plate with different blocking reagents (1% gelatin, 3% gelatin, or PBS);

FIG. 7(a) shows a plot of the raw ECL signal obtained for an MSD assay at different concentrations of IMP321 for different seeding densities of Raji cells; FIG. 7(b) shows a plot of the specific ECL signal obtained for an MSD assay at different concentrations of IMP321 for different seeding densities of Raji cells;

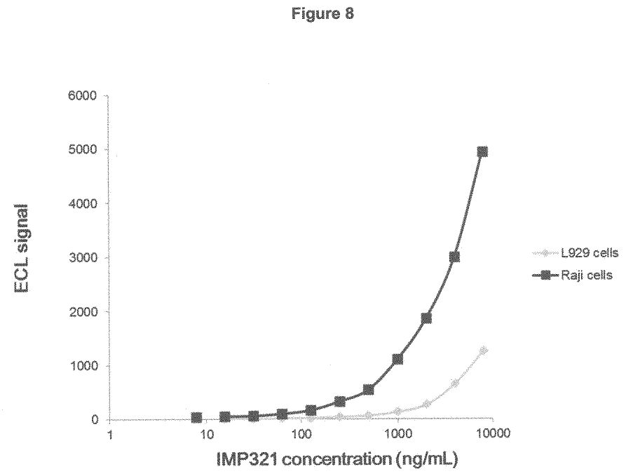

FIG. 8 shows a plot of the ECL signal obtained for an MSD assay for binding of different concentrations of IMP321 to Raji cells or HLA-DR^{dim} L929 cells following bocking of the MSD plate with casein:

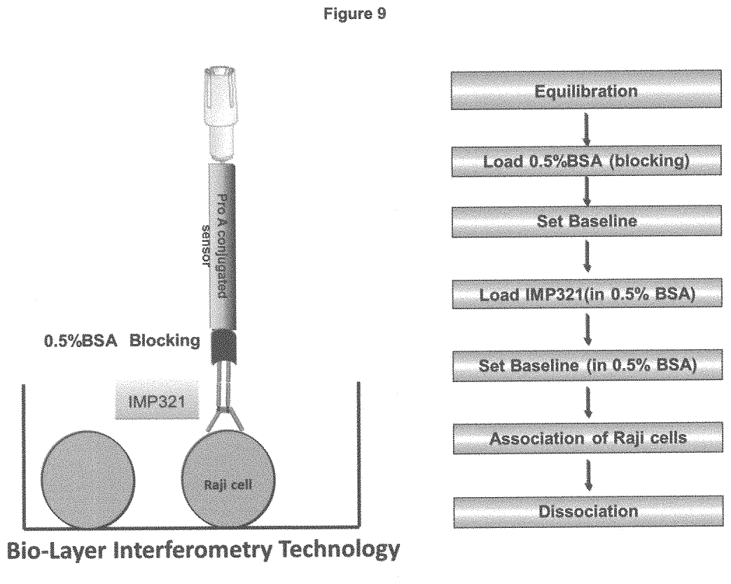

FIG. 9 shows schematically, on the left, a BLI probe with a protein A-conjugated sensor and IMP321 immobilised to the distal tip of the optical fibre of the sensor, with the tip of the sensor immersed in a sample solution containing Raji cells. The basic steps of the method are set out on the right of the figure;

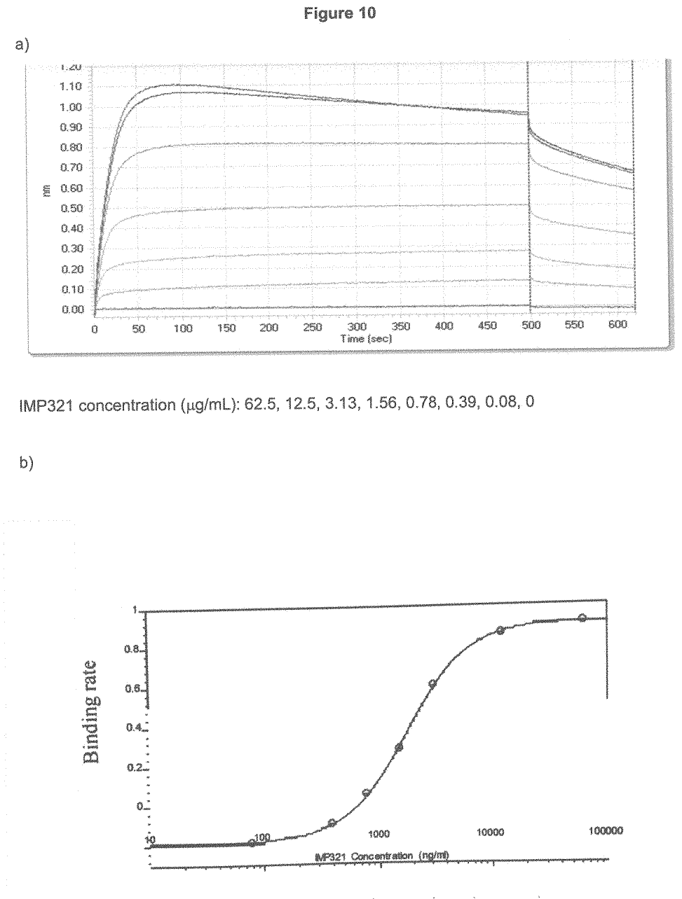

FIG. 10(a) shows a plot of the binding signal obtained in a BLI assay for dose-dependent binding of immobilised IMP321 to Raji cells in solution in the association step; FIG. 10(b) shows a standard curve of IMP321 dose-dependent binding to Raji cells in the BLI assay;

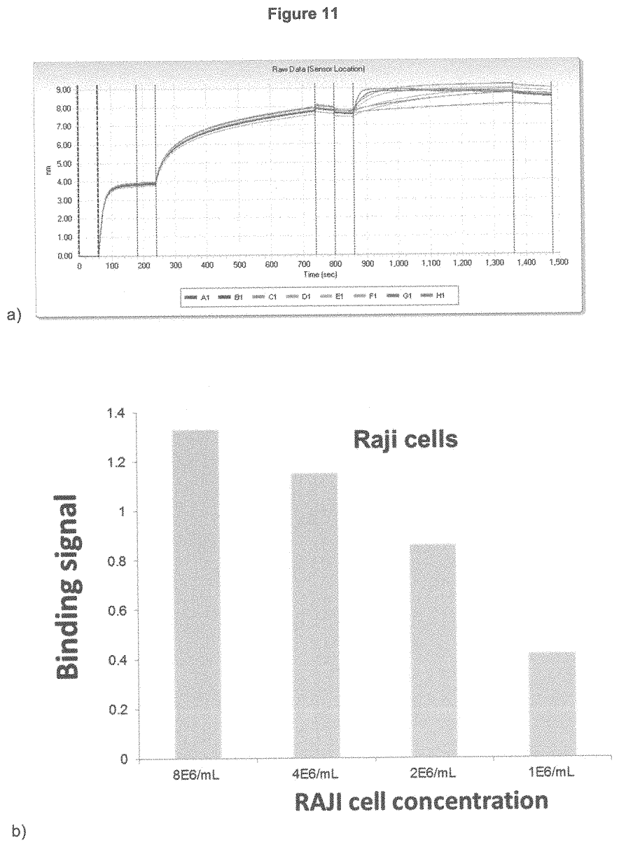

FIG. 11(a) shows the association and dissociation curves for binding of immobilised IMP321 to different concentrations of Raji cells (which are MHC class II-expressing) or Jurkat cells (which are not MHC class II-expressing) in solution in a BLI assay; FIG. 11(b) shows a graph of the binding signal obtained for the different Raji cell concentrations;

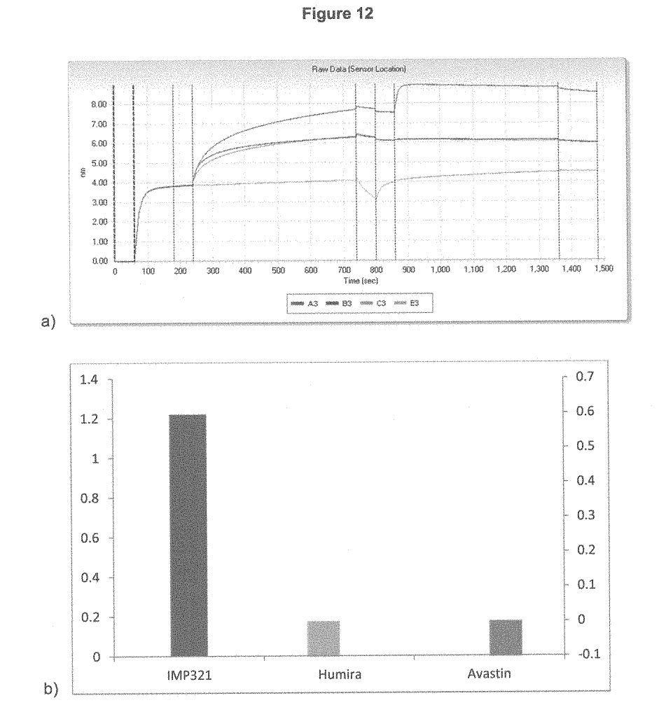

FIG. 12(a) shows the association and dissociation curves for binding of immobilised IMP321, Humira, or Avastin, to Raji cells in solution in a BLI assay; FIG. 12(b) shows a graph of the binding signal obtained for the different immobilised proteins;

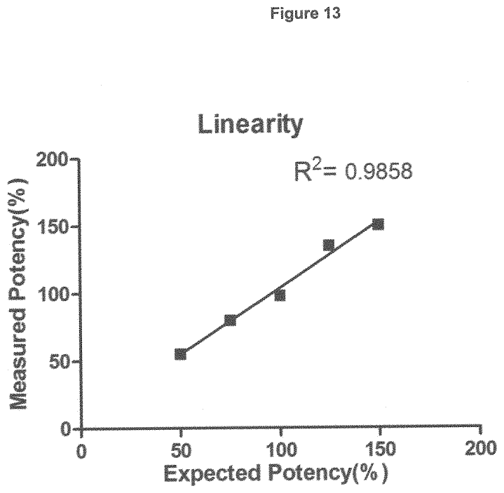

FIG. 13 shows a plot of the percentage binding potency, measured by BLI assay, for binding of different immobilised preparations of IMP321 to Raji cells in solution versus their expected potency;

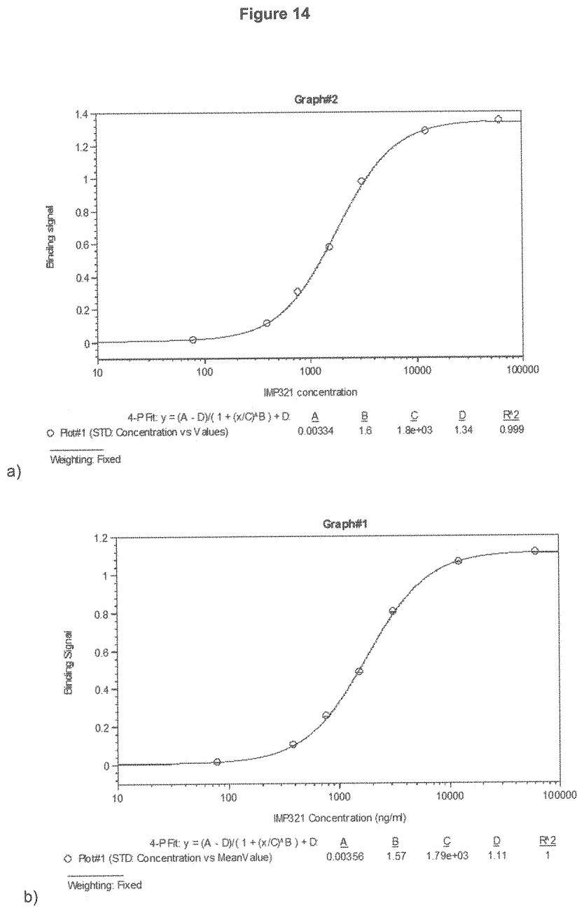

FIG. 14(a) shows a plot of the binding signal obtained by BLI assay for binding of different concentrations of immobilised IMP321 to previously cultured Raji cells in solution;

FIG. 14(b) shows a plot of the binding signal obtained by BLI assay for binding of different concentrations of immobilised IMP321 to previously frozen Raji cells in solution;

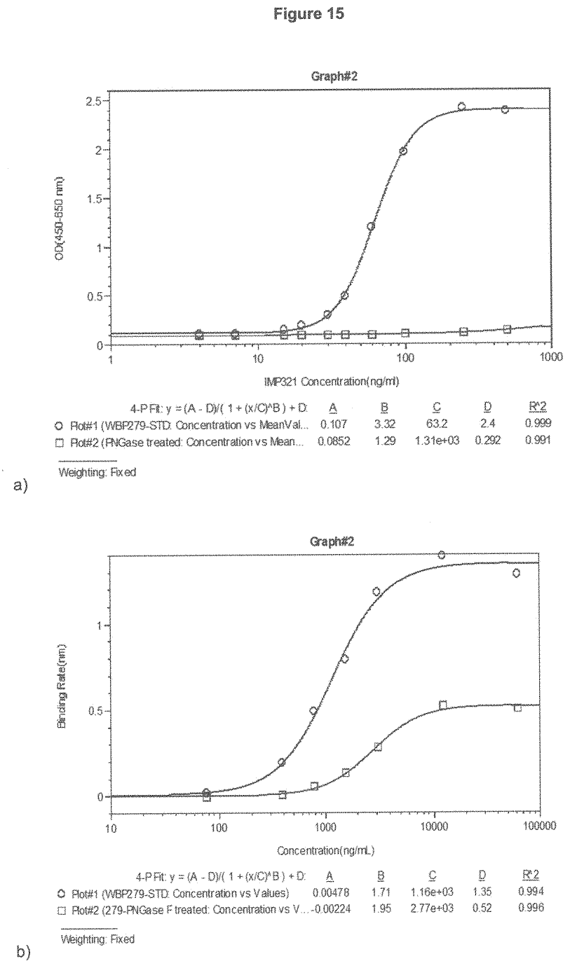

FIG. 15(a) shows a plot of the downstream CCL4 release obtained by cell-based assay for binding of different concentrations of immobilised IMP321, or deglycosylated IMP321, to Raji cells;

FIG. 15(b) shows a plot of the binding signal obtained by BLI assay for binding of different concentrations of immobilised IMP321, or deglycosylated IMP321, to Raji cells;

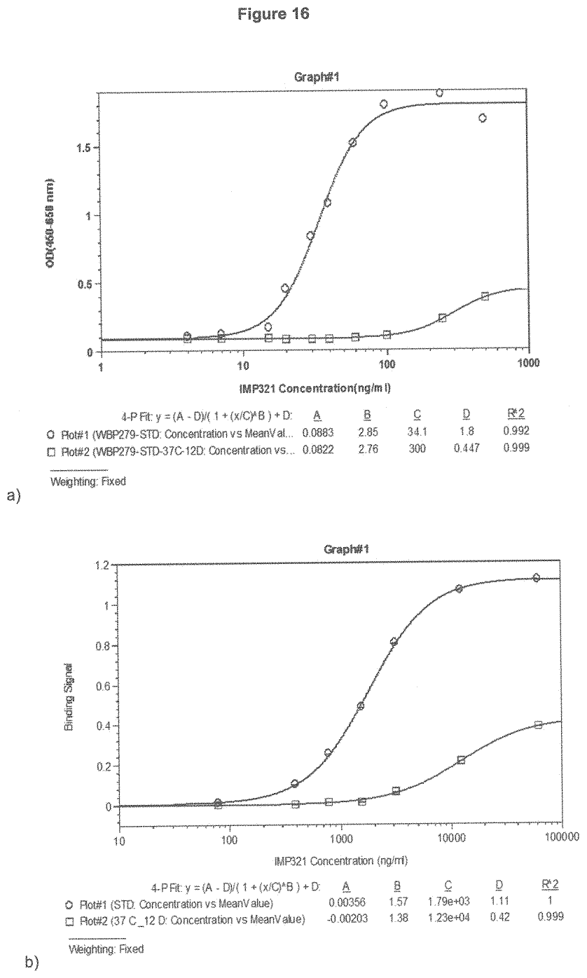

FIG. 16 shows plots of the signal for binding of different concentrations of immobilised IMP321, or IMP321 stored inappropriately (at 37° C. for 12 days) to Raji cells. The results shown in FIG. 16(a) were obtained by cell-based assay measuring CCL4 release, and the results shown in FIG. 16(b) were obtained by BLI assay;

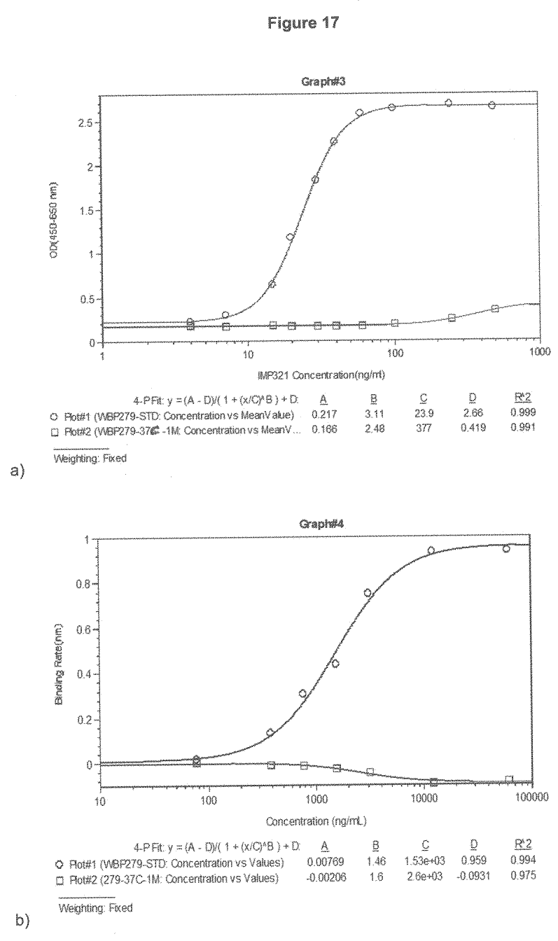

FIG. 17 shows plots of the signal for binding of different concentrations of immobilised IMP321, or IMP321 stored inappropriately (at 37° C. for 1 month) to Raji cells. The results shown in FIG. 17(a) were obtained by cell-based assay measuring CCL4 release, and the results shown in FIG. 17(b) were obtained by BLI assay;

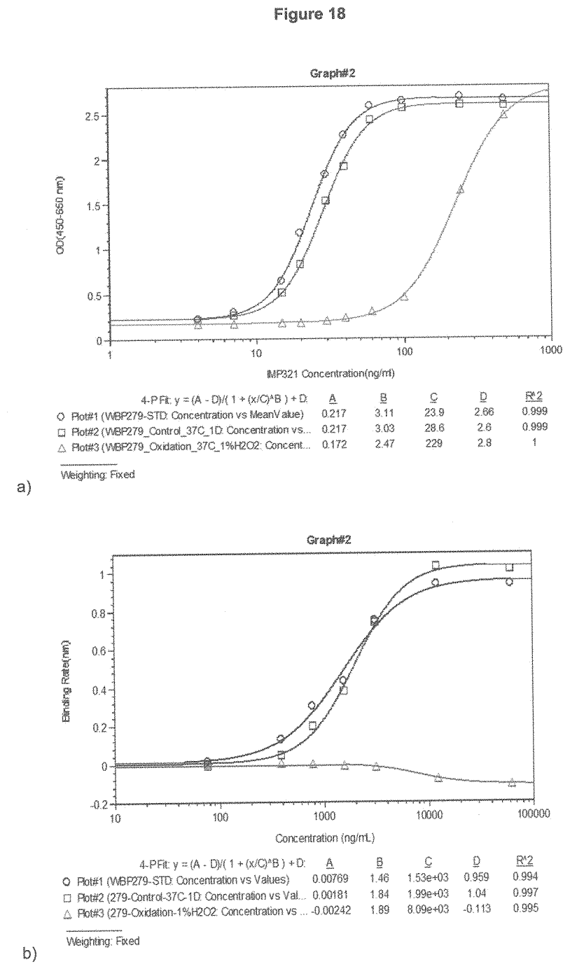

FIG. 18 shows plots of the signal obtained by cell-based assay measuring CCL4 release (FIG. 18a), or by BLI assay (FIG. 18b), for binding of different concentrations of immobilised IMP321 untreated, or oxidised IMP321 (with 1% hydrogen peroxide), to Raji cells;

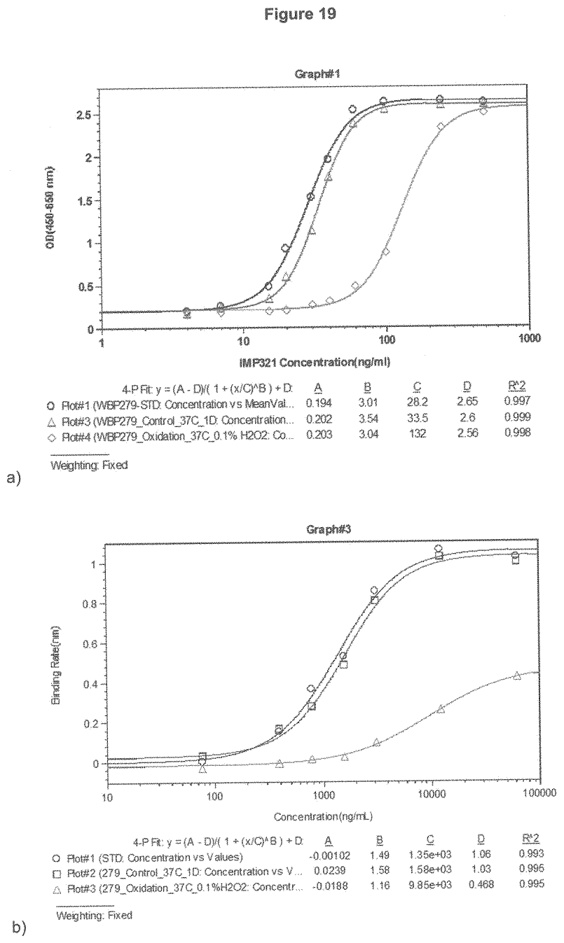

FIG. 19 shows plots of the signal obtained by cell-based assay measuring CCL4 release (FIG. 19a), or by BLI assay (FIG. 19b), for binding of different concentrations of immobilised IMP321 untreated, or oxidised IMP321 (with 0.1% hydrogen peroxide), to Raji cells;

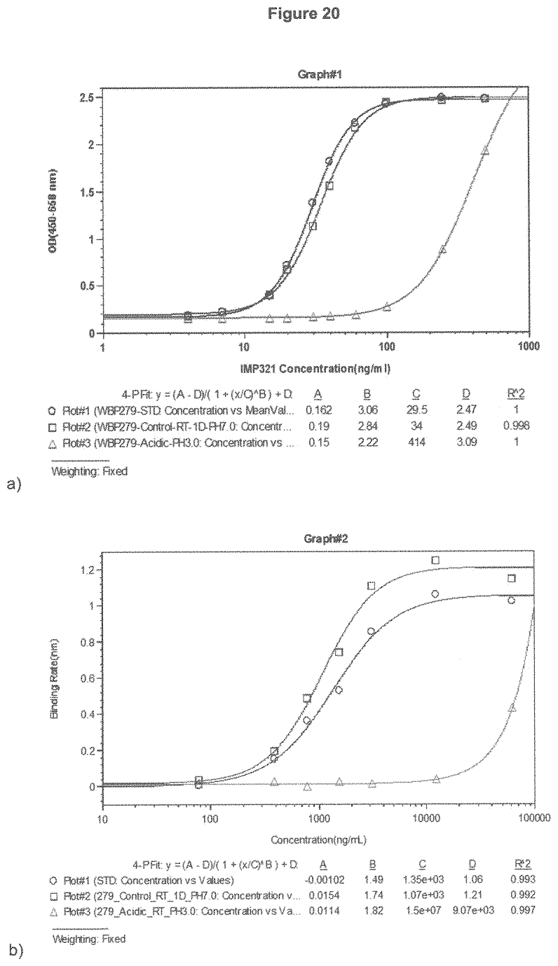

FIG. 20 shows plots of the signal obtained by cell-based assay measuring CCL4 release (FIG. 20a), or by BLI assay (FIG. 20b), for binding of different concentrations of immobilised IMP321 untreated, or acid-treated (at pH 3.0), to Raji cells;

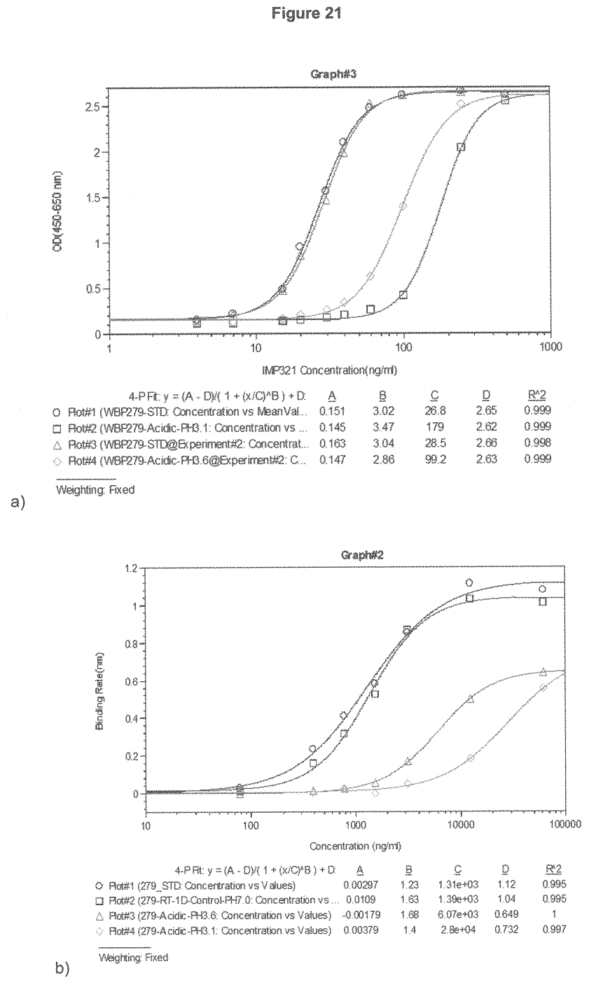

FIG. 21 shows plots of the signal obtained by cell-based assay measuring CCL4 release (FIG. 21a), or by BLI assay (FIG. 21b), for binding of different concentrations of immobilised IMP321 untreated, or acid-treated (at pH 3.1, or pH 3.6), to Raji cells;

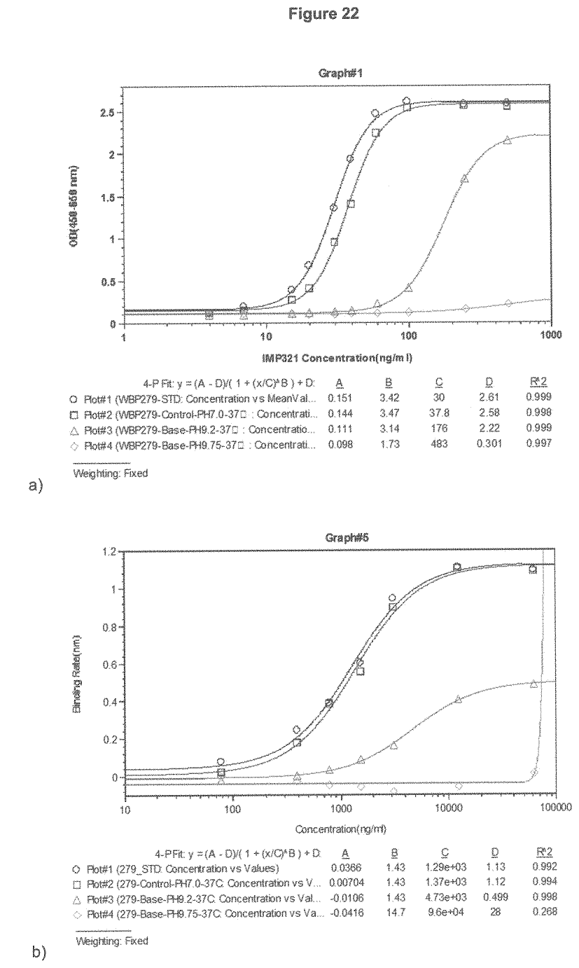

FIG. 22 shows plots of the signal obtained by cell-based assay measuring CCL4 release (FIG. 22a), or by BLI assay (FIG. 22b), for binding of different concentrations of immobilised IMP321 untreated, or base-treated (at pH 9.2 or pH 9.75), to Raji cells;

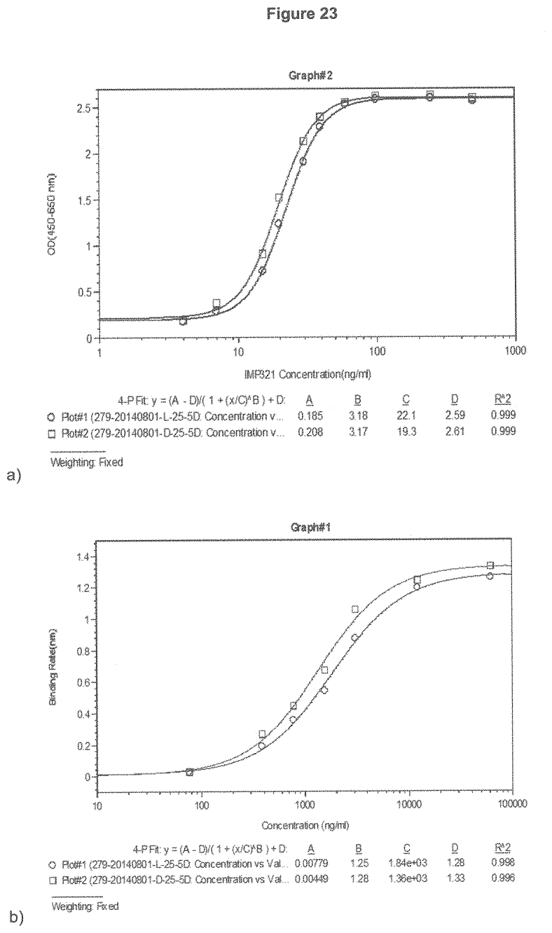

FIG. 23 shows plots of the signal obtained by cell-based assay measuring CCL4 release (FIG. 23a), or by BLI assay (FIG. 23b), for binding of different concentrations of immobilised IMP321 untreated, or light-exposed (at 25° C. for 5 days), to Raji cells;

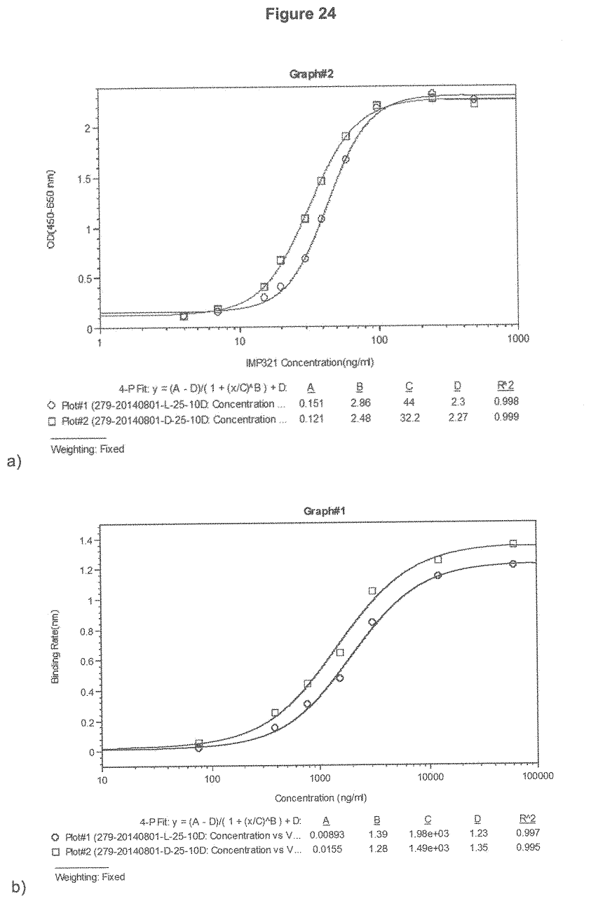

FIG. 24 shows plots of the signal obtained by cell-based assay measuring CCL4 release (FIG. 24a), or by BLI assay (FIG. 24b), for binding of different concentrations of immobilised IMP321 untreated, or light-exposed (at 25° C. for 10 days); and

FIG. 25 shows amino acid sequence of mature human LAG-3 protein. The four extracellular Ig superfamily domains are at amino acid residues: 1-149 (D1); 150-239 (D2); 240-330 (D3); and 331-412 (D4). The amino acid sequence of the extra-loop structure of the D1 domain of human LAG-3 protein is shown underlined in bold.

Examples 1 to 5 below describe evaluation of various different binding assays to determine whether they are suitable for use as quality control assays for GMP grade production of the recombinant LAG-3 protein derivative IMP321. None of the assays were found to be suitable. Examples 6 to 11 describe cell-based BLI methods, and demonstration of their suitability for determining MHC class II binding activity of preparations of IMP321.

### EXAMPLE 1

### Evaluation of Use of a Fluorescence-Activated Cell Sorting (FACS) Assay to Determine Binding of IMP321 to Raji Cells

A FACS assay was carried out to determine binding of IMP321 to Raji cells. IMP321 samples with 100%, 75%, and 50% MHC class II binding activity were tested. The sample with 100% activity was a reference sample with known MHC class II binding activity at a predetermined concentration. The samples with 75% and 50% activity were prepared by dilution of the reference sample.

The binding curves obtained are shown in FIG. 2. They show that no upper plateaus were reached, so there was no parallelism between the binding curve of the reference sample with 100% activity and the other samples. This prevented calculation of the relative potency of the different samples.

### EXAMPLE 2

### Evaluation of Use of a Meso Scale Discovery (MSD) Assay to Determine Binding of IMP321 to Raji Cells

This example describes evaluation of a Meso Scale Discovery (MSD) assay to determine binding of IMP321 to Raji cells.

The Meso Scale Discovery platform (MSD-ECL) uses electrochemiluminescent labels that are conjugated to detection antibodies. These labels generate light when stimulated by electricity in the appropriate chemical environment, which can then be used to measure key proteins and molecules.

Electricity is applied to the plate electrodes by the Meso Scale Discovery platform (MSD-ECL), leading to light emission by the labels. Light intensity is then measured to quantify analytes in the sample.

The detection process is initiated at electrodes located in the bottom of the Meso Scale Discovery (MSD-ECL)'s microplates, and only labels near the electrode are excited and detected. The system employs buffers with high concentrations of Tripropylamine as a catalyst for a dual redux reaction with Ruthenium, emitting light at 620 nm.

The MSD assay used is shown schematically in FIG. 3. Briefly, approximately 2×10⁴ cells per well of Raji cells in PBS were seeded into a Single-SPOT 96-well MSD plate (Meso Scale Discovery, Gaithersburg, Md.) at 25 uL/well. The plate was incubated at room temperature for 1-1.5 hours before being blocked with blocking buffer (25 uL/well). Then serial dilutions of IMP321 reference standard, or samples, were loaded into duplicate wells at 50 uL/well. After about 1 hour of incubation at room temperature, bound IMP321 was detected using ruthenium-conjugated anti-human Fc at 50 uL/well.

Electrochemiluminescence signal was acquired using MSD read buffer without surfactant. ECL counts should be proportional to IMP321 binding onto the cell surface within the assay range.

High binding carbon electrodes in the bottom of microplates allow for easy attachment of Raji cells. The assay uses electrochemiluminescent labels that are conjugated to anti-IMP321 antibodies. Electricity is applied to the plate electrodes by an MSD instrument leading to light emission by the labels. Light intensity is then measured to quantify the presence of IMP321 bound to MHC class molecules on the surface of the immobilised Raji cells.

The results obtained for samples containing IMP321 with and without Raji cells are shown in FIG. 4(a), and for samples containing Rituxan with and without Raji cells, is shown in FIG. 4(b).

The results show that non-specific binding of IMP321 to MSD plates was observed in the absence of Raji cells. By comparison, specific binding of Rituxan to Raji cells was observed.

Raji cells are cells of a cell line derived from the B-lymphocyte of an 11-year-old Nigerian Burkitt's lymphoma male patient in 1963. Rituxan (Rituximab) is a chimeric monoclonal antibody against the protein CD20, which is primarily found on the surface of B cells.

### EXAMPLE 3

### Evaluation of Non-Specific Binding of IMP321 to ELISA Plates

This example describes evaluation of non-specific binding of IMP321 and Rituxan to plates used for Enzyme-Linked Immunosorbent Assays (ELISAs) using different blocking reagents.

Briefly, microplates were blocked with blocking reagent at 25° C. for 2 hours. Samples and rituxan control were diluted with dilution buffer to 2 μg/ml then further diluted by two-fold serial dilution. Microplates were washed and well-drained before and after adding the diluted samples and incubation. After incubation with secondary antibody, the signal was measured by a spectrometry assay using SpectraMax M2 (450-650 nm).

| | Condition |
|---|---|
| Plate | ELISA plate (Costar) |
| Coating reagent | None |
| Blocking reagent/dilution buffer | 5% BSA in RPIM 1640 Medium |
| | 30% FBS in PBS/10% FBS in PBS |
| IMP321 or Rituxan concentration | 0~2 μg/ml |

The results are shown in FIG. 5. FIG. 5(a) shows the results of ELISA using increasing concentrations of IMP321 and ELISA plates blocked with 5% BSA or 10% FBS. FIG. 5(b) shows the results of ELISA using increasing concentrations of IMP321 or Rituxan and ELISA plates blocked with 30% FBS in PBS. FIG. 5(c) shows the results of ELISA using increasing concentrations of IMP321 or Rituxan and ELISA plates blocked with 5% BSA in RPIM 1640.

The results show that there was severe non-specific binding of IMP321, but not Rituxan, to ELISA plates when using BSA or FBS as blocking reagents.

Various different types of blocking agents were then tested with IMP321 or Rituxan to see if the non-specific binding of IMP321 to ELISA plates could be eliminated.

| | Condition |
|---|---|
| Plate | ELISA high bind plate |
| Coating reagent | None |
| Blocking reagent/dilution buffer | 1% non-fat milk in PBS |
| | 3% non-fat milk in PBS |
| | Blocker Casein Blocking Buffers |
| | (Thermo) |
| | 1% gelatin (from bovine skin) |
| | 3% gelatin (from bovine skin) |
| | PBS (control) |
| IMP321 or Rituxan concentration | 0~8 μg/ml |

The results are shown in FIG. 6. FIG. 6(a) shows the results for IMP321 or Rituxan using 1% non-fat milk, 3% non-fat milk, or Blocker Casein Blocking Buffers (Thermo) as blocking reagent. FIG. 6(b) shows the results for IMP321 or Rituxan using 1% gelatin, 3% gelatin, or PBS as blocking reagent.

The results show that Casein was the best blocking reagent to reduce non-specific binding of IMP321 to ELISA plates.

### EXAMPLE 4

Evaluation of Use of Meso Scale Discovery (MSD) Assay, with Casein Blocking Buffer, to Determine Binding of IMP321 to Raji Cells

This example describes evaluation of an MSD assay to determine binding of IMP321 to Raji cells at different seeding densities using casein blocking buffer.

An MSD assay was carried out, similar to that described in Example 2, to evaluate whether the non-specific binding of IMP321 to the MSD plate observed in that example could be minimized using Casein blocking buffer.

| | Condition |
|---|---|
| Plate | MSD high bind plate |
| Cell density | 5E4/well, 2.5E4/well, 5E3/well, 1E3/well |
| Blocking reagent/dilution | Blocker Casein Blocking Buffers (Thermo) |
| buffer | |
| IMP321 concentration | 0~8 μg/ml in casein blocking buffer |
| Goat anti-human antibody, | 500 ng/ml in casein blocking buffer |
| SULFO-TAG labelled | |

The results are shown in FIG. 7. FIG. 7(a) shows the results of binding of IMP321 to different seeding densities of Raji cells (0-5×10⁴ cells/well) at different concentrations of IMP321. The results show a cell density-dependent increase of maximal IMP321 binding. FIG. 7(b) shows the results of specific binding of IMP321 to different seeding densities of Raji cells (1×10³-5×10⁴ cells/well). The results show a cell density-dependent increase of specific IMP321 binding.

Binding of IMP321 to Raji cells was compared with binding of IMP321 to HLA-DR^{dim} L929 cells (these cells do not express MHC class II), at different concentrations of IMP321, using the MSD assay with casein blocking buffer. L929 is a fibroblast-like cell line cloned from strain L. The results are shown in FIG. 8. The results show that non-specific binding of IMP321 to MSD plates was significantly reduced in the presence of casein blocker. However, the specific binding signal was low, and no upper plateau of the IMP321 dose-binding curve was observed.

It was concluded that the MSD assay using casein blocking buffer cannot be used to demonstrate specific binding of IMP321 to plate-immobilised Raji cells.

### EXAMPLE 5

### Evaluation of Use of ELISA Assays to Determine Binding of IMP321 to Raji Cells

This example describes an evaluation of the ability of cell-based direct ELISA and cell-based transfer ELISA to determine binding of IMP321 to Raji cells.

Direct ELISA (similar to the assay described in Example 3) was carried out in the presence of different blocking reagents (5% BSA, 10% FBS, 0.5% Casein, or 3% gelatin) with different amounts of plate-immobilised Raji cells (10,000, 5,000, or 2,500 cells), and different concentrations of IMP321 or IMP321 treated with Peptide-N-Glycosidase F (PNGase F, an amidase that cleaves between the innermost GlcNAc and asparagine residues of high mannose, hybrid, and complex oligosaccharides from N-linked glycoproteins). The conditions used for the direct ELISA assay are summarised in the tables below:

| Culture plate wells | Conditions |
|---|---|
| 1 A-G | 5% BSA, PNGase IMP321, 10,000 cells |
| 2 A-G | 10% FBS, PNGase IMP321, 10,000 cells |
| 3 A-G | 5% BSA, PNGase IMP321, 5,000 cells |
| 4 A-G | 10% FBS, PNGase IMP321, 5,000 cells |
| 5 A-G | 5% BSA, PNGase IMP321, 2,500 cells |
| 6 A-G | 10% FBS, PNGase IMP321, 2,500 cells |
| 7 A-G | 0.5% Casein, IMP321, 10,000 cells |
| 8 A-G | 3% gelatin, IMP321, 10,000 cells |
| 9 A-G | 0.5% Casein, IMP321, 5,000 cells |
| 10 A-G | 3% gelatin, IMP321, 5,000 cells |
| 11 A-G | 0.5% Casein, IMP321, 2,500 cells |
| 12 A-G | 3% gelatin, IMP321, 2,500 cells |
| H 1-12 | No blocking reagent (NSB) |

| Culture plate wells | IMP321 concentration (ng/ml) |
|---|---|
| A 1-12 | 1000 |
| B 1-12 | 500 |
| C 1-12 | 250 |
| D 1-12 | 125 |
| E 1-12 | 62.5 |
| F 1-12 | 31.25 |
| G 1-12 | 15.625 |
| H 1-12 | 0 |

The results are shown in the table below.

| | 1 | 2 | 3 | 4 | 5 | 6 | 7 | 8 | 9 | 10 | 11 | 12 |
|---|---|---|---|---|---|---|---|---|---|---|---|---|
| A | 1.771 | 1.345 | 1.812 | 2.254 | 1.834 | 2.394 | 0.160 | 1.970 | 0.136 | 2.306 | 0.112 | 2.596 |
| B | 1.298 | 1.346 | 1.357 | 1.632 | 1.320 | 1.796 | 0.188 | 1.554 | 0.206 | 1.881 | 0.180 | 2.565 |
| C | 0.910 | 0.762 | 1.090 | 0.939 | 0.893 | 1.287 | 0.241 | 1.392 | 0.255 | 2.096 | 0.231 | 1.947 |
| D | 0.395 | 0.377 | 0.476 | 0.443 | 0.415 | 1.124 | 0.176 | 0.688 | 0.197 | 0.823 | 0.163 | 0.770 |
| E | 0.283 | 0.290 | 0.303 | 0.280 | 0.297 | 0.329 | 0.142 | 0.417 | 0.131 | 0.464 | 0.135 | 0.399 |
| F | 0.310 | 0.279 | 0.297 | 0.304 | 0.304 | 0.294 | 0.180 | 0.389 | 0.143 | 0.359 | 0.149 | 0.303 |
| G | 0.366 | 0.333 | 0.274 | 0.327 | 0.281 | 0.270 | 0.185 | 0.361 | 0.207 | 0.316 | 0.168 | 0.295 |
| H | 0.342 | 0.286 | 0.350 | 0.360 | 0.370 | 0.318 | 0.190 | 0.337 | 0.200 | 0.305 | 0.197 | 0.276 |

The results show dose-dependent IMP321 binding to plate-immobilised Raji cells.

To check whether IMP321 binds non-specifically to the ELISA plates, a direct ELISA was carried out in the absence of Raji cells, under the conditions summarised in the table below:

| Culture plate wells | Conditions |
|---|---|
| 1 A-G | 5% BSA, PNGase IMP321 |
| 2 A-G | 10% FBS, PNGase IMP321 |
| 3 A-G | 0.5% Casein, IMP321 |
| 4 A-G | 3% gelatin, IMP321 |
| H 1-4 | No blocking reaegent (NSB) |

The results are shown in the table below:

| | 1 | 2 | 3 | 4 |
|---|---|---|---|---|
| A | 1.898 | 2.625 | 1.056 | 2.587 |
| B | 1.808 | 2.607 | 1.960 | 2.527 |
| C | 1.626 | 2.443 | 2.326 | 2.400 |
| D | 1.381 | 1.526 | 1.101 | 1.710 |
| E | 0.857 | 0.896 | 0.342 | 0.856 |
| F | 0.388 | 0.419 | 0.193 | 0.375 |
| G | 0.300 | 0.302 | 0.128 | 0.245 |
| H | 0.210 | 0.129 | 0.143 | 0.104 |

The results show strong non-specific binding of IMP321 to the ELISA plate in the absence of plate-immobilised Raji cells. Neither casein nor gelatin blocking reagents, nor PNGase treatment of IMP321, removed the non-specific binding.

It was concluded that a direct cell-based ELISA cannot be used to demonstrate specific binding of IMP321 to plate-immobilised Raji cells.

A transfer cell ELISA was carried out to determine binding of different concentrations of IMP321, or IMP321 treated with PNGase, to immobilised Raji cells. Raji cells were transferred to another plate after binding to IMP321 or treated IMP321. The conditions used for the assay are summarised in the tables below.

| Culture plate wells | Conditions |
|---|---|
| B 1-12 | Raji cells and WT IMP321 |
| C 1-12 | Raji cells and treated IMP321 |
| D 1-12 | Raji cells and treated IMP321 |
| F 1-12 | No cells and WT IMP321 |
| G 1-12 | No cells and treated IMP321 |
| H 1-12 | No cells and treated IMP321 |

| | WT or treated IMP321 concentration |
|---|---|
| Culture plate wells | (ng/ml) |
| 1 B-D, F-H | 1000 |
| 2 B-D, F-H | 500 |
| 3 B-D, F-H | 250 |
| 4 B-D, F-H | 125 |
| 5 B-D, F-H | 62.5 |
| 6 B-D, F-H | 31.25 |
| 7 B-D, F-H | 15.63 |
| 8 B-D, F-H | 7.813 |
| 9 B-D, F-H | 3.906 |
| 10 B-D, F-H | 1.953 |
| 11 B-D, F-H | 0.977 |
| 12 B-D, F-H | 0 |

The results are shown in the table below:

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | 2.031 | 1.815 | 0.956 | 0.475 | 0.223 | 0.159 | 0.098 | 0.053 | 0.034 | 0.026 | 0.018 | 0.009 |
| C | 0.878 | 0.642 | 0.276 | 0.166 | 0.082 | 0.042 | 0.035 | 0.020 | 0.012 | 0.011 | 0.010 | 0.010 |
| D | 1.129 | 0.594 | 0.361 | 0.206 | 0.076 | 0.049 | 0.029 | 0.020 | 0.012 | 0.010 | 0.010 | 0.011 |
| E | | | | | | | | | | | | |
| F | 0.011 | 0.010 | 0.015 | 0.008 | 0.007 | 0.007 | 0.006 | 0.007 | 0.007 | 0.006 | 0.007 | 0.008 |
| G | 0.011 | 0.010 | 0.008 | 0.007 | 0.007 | 0.008 | 0.007 | 0.007 | 0.007 | 0.008 | 0.008 | 0.008 |
| H | 0.013 | 0.011 | 0.009 | 0.009 | 0.009 | 0.008 | 0.008 | 0.009 | 0.008 | 0.008 | 0.006 | 0.009 |

The results show that the well-to-well signal variation is not acceptable for a quality control method. The method is also labour-intensive. It was concluded that a cell-based transfer ELISA cannot be used to demonstrate specific binding of IMP321 to plate-immobilised Raji cells.

### EXAMPLE 6

### A Cell-Based Assay to Measure the Binding Activity of a Preparation of the LAG-3 Protein Derivative IMP321 Using Bio-Layer Interferometry (BLI)

IMP321 is a soluble recombinant derivative of LAG-3 protein with high affinity to MHC class II molecules. This example describes a cell-based assay to measure the binding activity of IMP321 to MHC class II-expressing Raji cells using BLI. The assay is simple and quick, and allows comparison between reference standards and samples.

FIG. 9 shows schematically, on the left, a BLI probe with a protein A-conjugated sensor and IMP321 immobilised to the distal tip of the optical fibre of the sensor, with the tip of the sensor immersed in a sample solution containing Raji cells. The basic steps of the method are set out on the right of the figure. The assay is described in more detail below.

### Materials:

1) Raji cells: ATCC/CCL-86

### 2) RPMI 1640: Invitrogen/22400-089

### 3) HI-FBS: Invitrogen/10100147

### 4) DPBS: Hyclone/SH30028.01B

### 5) BSA: Sigma/A3032

### 6) IMP321 Reference Material

### 7) Raji Cell Growth Medium: RPMI 1640, 10% HI-FBS

### 8) Binding Assay Diluent: DPBS, 0.5% BSA

### 9) Protein A Tray (ForteBio-18-5010)

10) 96-flat-bottom-well black plate (Greiner-655209)
11) Single- and multi-channel pipettes: Sartorius and Eppendorf/various
12) Cell counter: Roche/Cedex HiRes and Beckman/ViCell
13) Bio-Layer Interferometer: Fortebio/Octet Red with software version 7.0 or later

### Methods:

### 1. Preparation of Ready-to-Use Raji Cells

1) Remove N vial(s) of Raji cells from the liquid nitrogen freezer and quickly thaw in a 37° C. water bath.
2) Aseptically transfer the vial contents to a sterile centrifuge tube containing approximately N×9 mL of Raji Cell Growth Media. Mix well by gently pipetting.
3) Centrifuge the cells 5 min at 300×g. Resuspend cells in Binding Assay Diluent and count them with a cell counter or a hemacytometer.
4) Add the volume of cell stock suspension to a sufficient volume of Binding Assay Diluent to adjust cell densities to 4.0E6-8.0E6 cells per mL and keep on ice for use.

### 2. Preparation of IMP321 Reference Standard, Control and Samples

NOTE: 1) Use reverse pipetting to ensure accuracy.
2) Vortex gently to avoid or minimize creating foam and bubbles
1) Reference standard preparation:
1.1) Thaw a vial of IMP321 Reference Material as needed. Store at 2-8° C. Expiration is 7 days from date of thaw
1.2) Dilute IMP321 Reference Material to approximately 1.0 mg/mL in Formulation Buffer. Prepare fresh and use fresh. Determine the protein concentration spectrophotometrically using Formulation Buffer as a blank.
1.3) Based on measured protein concentration, dilute RM to prepare standard curve to the appropriate concentrations as described below. Mix dilutions by votexing.

| | | | Volume of | Volume of Assay |
|---|---|---|---|---|
| Tube | IMP321 concentration | | IMP321 Dilution | Diluent |
| A | ~30 | mg/mL | — | — |
| B | ~1.0 | mg/mL | 40 μL of A | 1160 μL |
| C | 62.5 | μg/mL | 40 μL of B | XXX mL |
| D | 12.5 | μg/mL | 400 μL of C | 1600 μL |
| E | 3125 | ng/mL | 400 μL of D | 1200 μL |
| F | 1562.5 | ng/mL | 200 μL of D | 1400 μL |
| G | 781.25 | ng/mL | 100 μL of D | 1500 μL |
| H | 390.625 | ng/mL | 50 μL of D | 1550 μL |
| I | 78.125 | ng/mL | 400 μL of H | 1600 μL |

| | | | |
|---|---|---|---|
| J | 0 | — | 1000 μL |

1.4) Use dilutions C-J for the standard curve. Additional concentrations may be used if needed, to include the linear portion of the curve and the upper and lower plateaus.
2) Preparation of Control
2.1) The control is an independent dilution of Reference Material from Tube C prepared in Step 1.3 above. Further dilute as described in the Table above. Mix dilutions by votexing.
2.2) Use dilutions C-J for the Control.
3) Preparation of Samples
3.1) Based on protein concentration, dilute IMP321 Samples to approximately 1.0 mg/mL in Assay Diluent. Prepare fresh and use fresh.
3.2) Further dilute to prepare standard curve to the appropriate concentrations as described in the Table above. Mix dilutions by votexing.
3.3) Use dilutions C-J for the Samples. Additional concentrations may be used if needed, to include the linear portion of the curve and the upper and lower plateaus.

### 3. Detection Steps in the Octet System

1) Hydrate the biosensors in PBS for at least 10 min
2) Prepare the assay plate. In a black polypropylene microplate, transfer 200 μL per well of PBS, Assay Diluent, titrations of IMP321 in AD, or Raji cells respectively into the appropriate wells according to the Sample Plate Map below:

Sample Plate Map

| | 1 | 2 | 3 | 4 | 5 | 6 | 7 | 8 | 9 | 10 | 11 | 12 |
|---|---|---|---|---|---|---|---|---|---|---|---|---|
| A | B | L | B | L | B | S | B | E | E | E | E | E |
| B | B | L | B | L | B | S | B | E | E | E | E | E |
| C | B | L | B | L | B | S | B | E | E | E | E | E |
| D | B | L | B | L | B | S | B | E | E | E | E | E |
| E | B | L | B | L | B | S | B | E | E | E | E | E |
| F | B | L | B | L | B | S | B | E | E | E | E | E |
| G | B | L | B | L | B | S | B | E | E | E | E | E |
| H | B | L | B | B | B | S | B | E | E | E | E | E |

| 1 | 2 | 3 | 4 | 5 | 6 | 7 |
|---|---|---|---|---|---|---|
| DPBS | DA | DA | Sample | DA | Cell | DA |

B = Buffer
S = Sample
L = Loading
E = Empty

3) Set up a kinetic assay with the parameter settings described below.
4) Enter location and file name for saving the data.
5) Click GO to run the assay.

| Assay | Step Data | Sample | | |
|---|---|---|---|---|
| Step Number | Name | Column | Step Type | Assay Time(s) |
| 1 | Equilibration | 1 | Custom | 60 |
| 2 | Loading | 2 | Loading | 120 |
| 3 | Baseline | 3 | Baseline | 60 |
| 4 | Loading2 | 4 | Loading | 500 |
| 5 | Baseline | 5 | Baseline | 60 |
| 6 | Association | 6 | Association | 500 |
| 7 | Dissociation | 7 | Dissociation | 120 |

### 4. Analyze Data

1) In the Octet Data Analysis software, load the data folder to be analyzed.
2) In the Processing tab, select Association step. Then click on the “quantitate the Selected Step”.
3) Input Concentration information accordingly.
4) In the Results tab, select R equilibrium (Req) as the binding rate equation. This equation will fit the binding curve generated during the experiment and calculate a response at equilibrium as the output signal.
5) Click on Calculate Binding Rate. Results will be displayed automatically in the table.
6) Click the Save Report button to generate a MS Excel report file.
7) Use SoftMax Pro, a 4-parameter logistic curve-fitting program, to generate a standard curve or sample curve by Binding rate (nm) against the IMP321 concentration expressed ug/mL. An example is shown in FIG. 10.
8) Calculate relative binding potency of the sample using EC50 ratio of the Reference Standard and the Sample.

### 5. System Suitability and Assay Acceptance Criteria.

An assay is valid if it meets ALL following criteria:

1) Ready to use Raji cell viability>=60%
2) Relative activity of the control is within 80%-120%
3) Signal to Background ratio of the control (Parameter D/Parameter A)>=2.
4) Parallelism (comparability): slope ratio with the Standard is between 0.8 and 1.4.
5) If the result for the assay control does not meet the criteria listed above, the assay is considered invalid.

### 6. Reportable Value:

1) For a clinical sample, the reportable value for a sample is defined as the mean of two or three valid and independent assay results as detailed below:
% Difference is calculated as follows:

Absolute value (Assay 1 Result−Assay 2 Result)/Mean value (Assay 1 Result,Assay 2 Result)×100%

2) If the % Difference of the two assay results<=20%, report mean results of the two assays.
3) If the % Difference of the two assay results>20%, perform 1 additional valid assay.
4) If the CV of the three sample assay results<=25%, report mean results of the three assays.
5) If the CV of the three sample assay results>25%, there is no reportable value. Initiate a discrepancy with a re-test plan.
6) If the reportable value for a sample does not meet specifications listed in the COA, initiate a discrepancy with a retest plan.

### 7. Retest Plan

Perform the retest of a sample as follows:

1) Retest the sample with three valid and independent assays
2) If the CV of the three sample assay results<=25%, report mean results of the three assays.
3) If the CV of the three sample assay results>25%, there is no reportable value.
4) If the retest result is out of specification (OOS) listed in the COA, the conclusion is fail.

### EXAMPLE 7

### Determination of Specific Binding of Immobilised IMP321 to Raji Cells in Solution in a BLI Assay

A BLI assay as described in Example 6 was used to determine binding of immobilised IMP321 to different concentrations of Raji cells in solution (8E6/mL, 4E6/mL, 2E6/mL, 1E6/ml). Jurket cells were used as a negative control. The association and dissociation curves obtained are shown in FIG. 11(a). FIG. 11(b) shows a graph of the binding signal obtained for the different Raji cell concentrations. The results show that the binding signal was dependent on the concentration of Raji cells, i.e. the higher the concentration of Raji cells, the higher the binding rate and upper plateau obtained. No specific binding of Jurket cells was observed in the same assay.

A further BLI assay was performed as described in Example 6, but to compare binding of immobilised IMP321 to Raji cells with binding of immobilised Humira or Avastin. The association and dissociation curves obtained are shown in FIG. 12(a). FIG. 12(b) shows a graph of the binding signal obtained for the different immobilised proteins. The results show that IMP321, but not Humira or Avastin, binds to Raji cells.

It was concluded from these results that the BLI assay is able to determine specific binding of immobilised IMP321 to Raji cells in solution.

### EXAMPLE 8

Correlation of IMP321 Binding Activity Measured by BLI Assay with Known Binding Potency

Samples of IMP321 diluted from reference standard with different levels of Raji cell binding potency were used in a BLI assay to determine whether the binding activity measured by the assay correlated with the known binding potency of the samples. The results are shown in the table below. FIG. 13 shows a plot of the percentage binding potency, measured by BLI assay, versus their expected potency;

| | Potency determined by | |
|---|---|---|
| Sample binding potency | BLI assay | Percentage recovery |
| 50% | 55% | 110% |
| 75% | 80% | 107% |
| 100% | 98% | 98% |
| 125% | 135% | 108% |
| 150% | 150% | 100% |

The results show a good correlation between the binding potency measured by BLI assay, and the expected binding potency. Mean recoveries of each sample were from 90% to 110%, with good parallelism of binding curves (i.e. acceptable slope ratio and converged plateaus).

### EXAMPLE 9

### Use of Frozen Cells in a BLI Assay to Determine MHC Class II Binding Activity

A BLI assay as described in Example 6 was carried out to compare binding of immobilised IMP321 to Raji cells in solution obtained from culture or from a frozen stock solution. A plot of the binding signal obtained for binding of different concentrations of immobilised IMP321 to cultured Raji cells in solution is shown in FIG. 14(a). A plot of the binding signal obtained for binding of different concentrations of immobilised IMP321 to previously frozen Raji cells in solution is shown in FIG. 14(b).

The results show that the frozen Raji cells behave very similarly to the cultured Raji cells, and so the frozen stock solution can be used in place of a fresh culture solution, thereby providing improved assay robustness and transferability.

### EXAMPLE 10

### In-Process Sample Testing

BLI assays as described in Example 6 were carried out to determine the MHC class II binding activity of various different preparations of IMP321, and to compare the bioactivity of the preparations as determined by CCL4 release assay.

THP-1 is a human single nuclear leukaemia cell line. When induced with LAG-3 protein, or stressed samples, THP-1 cells secrete cytokine CCL4 which can be quantified with a CCL4 ELISA kit. The level of CCL4 release can be used to measure the bioactivity of a preparation of LAG-3 protein, or a fragment, derivative, or analogue thereof.

| | Bioactivity | Bioactivity |
|---|---|---|
| IMP321 Sample | (CCL4 release) | (binding) |
| SD140817K01 | 102% | 92% |
| 20140801-T0 | 101% | 89% |
| 20140802-T0 | 102% | 91% |
| 20140801-T0-PC | 98% | 102% |
| 20140802-T0-PC | 97% | 91% |
| 20140801-D-25-5D | 104% | 93% |
| 20140802-D-25-5D | 96% | 87% |
| 20140803-T0 | 110% | 86% |
| 20140804-T0 | 104% | 100% |

It was concluded that the bioactivity of the different IMP321 samples correlated with the bioactivity as determined by CCL4 release assay.

### EXAMPLE 11

### BLI Assay Testing of Stressed IMP321 Samples and Correlation to a Cell-Based CCL4 Release Assay

BLI assays as described in Example 6 were used to determine MHC class II binding activity of IMP321 samples that have been exposed to different treatments (deglycosylation by treatment with PNGase, storage at 37° C., oxidation by treatment with 1% or 0.1% hydrogen peroxide, treatment with acid at pH 3.0, 3.6, or 3.1, treatment with alkali at pH 9.2, 9.75, or exposure to light). The results are shown in FIGS. 15-24.

FIG. 15(a) shows a plot of the downstream CCL4 release obtained by cell-based assay for binding of different concentrations of immobilised IMP321, or deglycosylated IMP321, to Raji cells;

FIG. 15(b) shows a plot of the binding signal obtained by BLI assay for binding of different concentrations of immobilised IMP321, or deglycosylated IMP321, to Raji cells;

FIG. 16 shows plots of the signal for binding of different concentrations of immobilised IMP321, or IMP321 stored inappropriately (at 37° C. for 12 days) to Raji cells. The results shown in FIG. 16(a) were obtained by cell-based assay measuring CCL4 release, and the results shown in FIG. 16(b) were obtained by BLI assay;

FIG. 17 shows plots of the signal for binding of different concentrations of immobilised IMP321, or IMP321 stored inappropriately (at 37° C. for 1 month) to Raji cells. The results shown in FIG. 17(a) were obtained by cell-based assay measuring CCL4 release, and the results shown in FIG. 17(b) were obtained by BLI assay;

FIG. 18 shows plots of the signal obtained by cell-based assay measuring CCL4 release (FIG. 18a), or by BLI assay (FIG. 18b), for binding of different concentrations of immobilised IMP321 untreated, or oxidised IMP321 (with 1% hydrogen peroxide), to Raji cells;

FIG. 19 shows plots of the signal obtained by cell-based assay measuring CCL4 release (FIG. 19a), or by BLI assay (FIG. 19b), for binding of different concentrations of immobilised IMP321 untreated, or oxidised IMP321 (with 0.1% hydrogen peroxide), to Raji cells;

FIG. 20 shows plots of the signal obtained by cell-based assay measuring CCL4 release (FIG. 20a), or by BLI assay (FIG. 20b), for binding of different concentrations of immobilised IMP321 untreated, or acid-treated (at pH 3.0), to Raji cells;

FIG. 21 shows plots of the signal obtained by cell-based assay measuring CCL4 release (FIG. 21a), or by BLI assay (FIG. 21b), for binding of different concentrations of immobilised IMP321 untreated, or acid-treated (at pH 3.1, or pH 3.6), to Raji cells;

FIG. 22 shows plots of the signal obtained by cell-based assay measuring CCL4 release (FIG. 22a), or by BLI assay (FIG. 22b), for binding of different concentrations of immobilised IMP321 untreated, or base-treated (at pH 9.2 or pH 9.75), to Raji cells;

FIG. 23 shows plots of the signal obtained by cell-based assay measuring CCL4 release (FIG. 23a), or by BLI assay (FIG. 23b), for binding of different concentrations of immobilised IMP321 untreated, or light-exposed (at 25° C. for 5 days), to Raji cells; and

FIG. 24 shows plots of the signal obtained by cell-based assay measuring CCL4 release (FIG. 24a), or by BLI assay (FIG. 24b), for binding of different concentrations of immobilised IMP321 untreated, or light-exposed (at 25° C. for 10 days).

The bioactivity (as determined by CCL4 release of the different IMP321 samples, compared with their MHC class II binding activity (determined by a method as described in Example 6) is shown in the table below:

| FIG. | | Bioactivity | Bioactivity |
|---|---|---|---|
| No. | Sample | (CCL4 release) | (binding) |
| 15 | IMP321 PNGase treated | None | NRR |
| 16 | IMP321 stored at 37° C. (12 D = | NRR | NRR |
| | 12 days) | | |
| 17 | IMP321 stored at 37° C. (1 M = | None | None |
| | 1 month) | | |
| 18 | IMP321 stored at 37° C. (1 D = | 84% | 77% |
| | 1 day) Control | | |
| | IMP321 Oxidation, 1% H₂O₂ | 10% | None |
| | at 37° C. (1 D = 1 day) | | |
| 19 | IMP321 stored at 37° C. (1 D = | 84% | 85% |
| | 1 day) Control | | |
| | IMP321 Oxidation, 0.1% | 21% | NRR |
| | H₂O₂ at 37° C. (1 D = 1 day) | | |
| 20 | IMP321, pH 7.0 at RT (1 D = | 87% | 126% |
| | 1 day) | | |
| | IMP321, Acid pH 3.0 at RT | 7% | None |
| | (1 D = 1 day) | | |
| 21 | IMP321, pH 7.0 at RT (1 D = | NA | 95% |
| | 1 day) | | |
| | IMP321, Acid pH 3.6 at RT | 29% | NRR |
| | (1 D = 1 day) | | |
| | IMP321, Acid pH 3.1 at RT | 15% | NRR |
| | (1 D = 1 day) | | |
| 22 | IMP321, pH 7.0 at 37° C. (1 D = | 79% | 94% |
| | 1 day) | | |
| | IMP321, Alkali pH 9.2 at | 17% | NRR |
| | 37° C. (1 D = 1 day) | | |
| | IMP321, Alkali pH 9.75 at | None | None |
| | 37° C. (1 D = 1 day) | | |
| 23 | IMP321, Dark, 5 D = 5 days | 100% | 100% |
| | IMP321, Light, 5 D = 5 days | 87% | 74% |
| 24 | IMP321, Dark, 10 D = 10 days | 100% | 100% |
| | IMP321, Light, 10 D = 10 | 73% | 75% |
| | days | | |

The results show a good correlation between the bioactivity of each treated IMP321 sample, as determined by CCL4 release, and its MHC class II binding activity, as determined by BLI assay according to the invention. It was concluded that determination of MHC class II binding activity by BLI assay can be used to determine the bioactivity of IMP321 preparations.

1. A method for determining MHC class II binding activity of a preparation comprising lymphocyte activation gene-3 (LAG-3) protein, or a fragment, derivative, or analogue thereof, wherein the method comprises determining binding of the LAG-3 protein, fragment, derivative, or analogue to MHC class II molecules using bio-layer interferometry (BLI).

2. A method according to claim 1, which comprises determining binding of the LAG-3 protein, fragment, derivative, or analogue to MHC class II molecules present on MHC class II-expressing cells.

3. A method according to claim 2, wherein the LAG-3 protein, fragment, derivative, or analogue is immobilised to a reagent layer of a BLI probe, and the MHC class II-expressing cells are in solution.

4. A method according to claim 3, wherein the MHC class H-expressing cells are present at a density of at least 1E6/mL, preferably at least 4E6/mL or 8E6/mL.

5. A method according to claim 3 or 4, wherein the reagent layer has been pre-treated with a blocking reagent to minimise non-specific binding of the MHC class II-expressing cells to the reagent layer.

6. A method according to claim 5, wherein the blocking reagent comprises albumin, preferably bovine serum albumin (BSA).

7. A method according to any of claims 2 to 6, wherein the MHC class H-expressing cells are Raji cells.

8. A method according to any of claims 2 to 7, wherein the MHC class H-expressing cells are thawed, ready-to-use cells obtained from a frozen stock solution.

9. A method according to any preceding claim, which comprises determining a rate of binding of the LAG-3 protein, fragment, derivative, or analogue, to the MHC class II molecules for a plurality of different concentrations of the LAG-3 protein, fragment, derivative, or analogue, and generating a dose-response curve for the rates of binding.

10. A method according to any preceding claim, which further comprises determining MHC class H binding activity of a reference sample of LAG-3 protein, or a fragment, derivative, or analogue thereof, by determining binding of the LAG-3 protein, fragment, derivative, or analogue of the reference sample to MHC class H molecules using BLI, under the same conditions used for determining binding of the LAG-3 protein, fragment, derivative, or analogue of the preparation, and comparing the MHC class II binding activity determined for the reference sample with the MHC class II binding activity determined for the preparation.

11. A method according to claim 10, wherein the MHC class II binding activity of the reference sample is set at 100%.

12. A method according to claim 10 or 11, wherein the reference sample comprises a LAG-3 protein, or a fragment, derivative, or analogue thereof, that has been treated to reduce its MHC class II binding activity.

13. A method according to claim 12, wherein the LAG-3 protein, fragment, derivative, or analogue, of the reference sample has been deglycosylated, stored at 370° C. for at least 12 days, oxidised, denatured by acid or alkali treatment, or exposed to light for at least 5 days.

14. A BLI probe for determining MHC class II binding activity of LAG-3 protein, or a fragment, derivative, or analogue thereof, which comprises a reagent layer to which the LAG-3 protein, or fragment, derivative, or analogue thereof, is immobilised.

15. A probe according to claim 14, wherein the reagent layer has been pre-treated with a blocking reagent to minimise non-specific binding of the MHC class H-expressing cells to the reagent layer.

16. A probe according to claim 15, wherein the blocking reagent comprises albumin, preferably BSA.

17. A kit for determining MHC class II binding activity of LAG-3 protein, or a fragment, derivative, or analogue thereof, which comprises a BLI probe having reagent layer to which the LAG-3 protein, or fragment, derivative, or analogue thereof, is immobilised, and MHC class II-expressing cells.

18. A kit according to claim 17, wherein the reagent layer of the BLI probe has been pre-treated with a blocking reagent to minimise non-specific binding of the MHC class H-expressing cells to the reagent layer.

19. A kit according to claim 18, wherein the blocking reagent comprises albumin, preferably BSA.

20. A kit according to any of claims 17 to 19, wherein the MHC class H-expressing cells are frozen cells.

21. A kit according to any of claims 17 to 20, wherein the cells are Raji cells.

22. A kit according to any of claims 17 to 21, wherein the cells are present at a density of at least 1E6/mL, preferably at least 4E6/mL or 8E6/mL.

23. A kit according to any of claims 17 to 22, which further includes a reference sample comprising LAG-3 protein, or a fragment, derivative, or analogue thereof.

24. A kit according to claim 23, wherein the MHC class H binding activity of the reference sample is known.
